(19) United States
(12) Patent Application Publication
(10) Pub. No.: US 2024/0000925 A1
(43) Pub. Date: Jan. 4, 2024

(54) ALLERGEN-SPECIFIC IMMUNOTHERAPY

(71) Applicant: Allergy Therapeutics (UK) Limited, Worthing (GB)

(72) Inventors: Pieter-Jan De Kam, Wageningen (NL); Matthew D. Heath, Brighton (GB); Matthias F. Kramer, Gauting (DE); Simon J. Hewings, Brighton (GB); Murray A. Skinner, Brighton (GB); Kemi Oluwayi, Maidstone (GB)

(21) Appl. No.: 18/217,095

(22) Filed: Jun. 30, 2023

Related U.S. Application Data
(60) Provisional application No. 63/357,736, filed on Jul. 1, 2022.

(51) Int. Cl. A61K 39/36; A61P 37/08

(52) U.S. Cl. CPC A61K 39/36; A61P 37/08; A61K 2039/545

(57) ABSTRACT

A method of treating allergic rhinitis and/or rhinoconjunctivitis in a subject, the method comprising administering to a subject in need thereof a therapeutically effective allergen-specific immunotherapy comprising or consisting of: (a) a build-up phase consisting of three weekly subcutaneous injections of a composition comprising one or more antigens; and (b) a maintenance phase consisting of three monthly subcutaneous injections of a composition comprising one or more antigens.

### CROSS-REFERENCE TO RELATED APPLICATIONS

This application claims priority to U.S. Provisional Application No. 63/357,736, filed Jul. 1, 2022, which is incorporated by reference herein in its entirety.

### FIELD OF THE INVENTION

The present invention relates to methods of treating allergic rhinitis and/or rhinoconjunctivitis in a subject, the methods comprising administering to a subject in need thereof a therapeutically effective allergen-specific immunotherapy. The present invention also relates to kits for treating allergic rhinitis and/or rhinoconjunctivitis in a subject.

### BACKGROUND TO THE INVENTION

Allergen-specific immunotherapy, is a medical treatment for environmental allergies that involves exposing a subject to increased doses of allergen in an attempt to change the immune system's response. Subcutaneous allergen-specific immunotherapy is generally safe and effective for allergic rhinitis, allergic rhinoconjunctivitis, allergic forms of asthma, and stinging insects.

Early vaccines used for allergen-specific immunotherapy were typically composed of aqueous extracts containing allergens from source materials to which people had an allergy, and consisted of increasing dose regimens which specifically desensitized the patient. Various local and occasional systemic adverse events were associated with the treatment. Formulations have been developed over the years to increase both the safety and efficacy of such treatment. However, relatively long treatment duration, high cost, and frequent hyper reactivity reactions, including anaphylaxis, mean that allergen-specific immunotherapy is not widely used in many countries.

There is a demand for new methods and kits for treating allergic rhinitis and/or rhinoconjunctivitis.

### SUMMARY OF THE INVENTION

The inventors have surprisingly found that an extended posology consistently demonstrated a more pronounced treatment effect compared to a conventional posology in a subcutaneous allergen-specific immunotherapy for treating subjects with allergic rhinitis and/or rhinoconjunctivitis.

In one aspect, the present invention provides a method of treating allergic rhinitis and/or rhinoconjunctivitis in a subject, the method comprising administering to a subject in need thereof a therapeutically effective allergen-specific immunotherapy comprising or consisting of: (a) a build-up phase consisting of three weekly subcutaneous injections of a composition comprising one or more antigens; and (b) a maintenance phase consisting of three monthly subcutaneous injections of a composition comprising one or more antigens.

In some embodiments, the build-up phase consists of: (i) a first weekly subcutaneous injection of a composition comprising one or more antigens in a dose of about 300 to about 1500 SU; (ii) a second weekly subcutaneous injection of a composition comprising one or more antigens in a dose of about 2000 to about 4000 SU; and (iii) a third weekly subcutaneous injection of a composition comprising one or more antigens in a dose of about 5000 to about 8000 SU. In some embodiments, the second weekly subcutaneous injection is administered about 6 to about 9 days after the first subcutaneous injection and the third weekly subcutaneous injection is administered about 6 to about 9 days after the second weekly subcutaneous injection. In some embodiments, the first weekly subcutaneous injection is a dose of about 900 SU, the second weekly subcutaneous injection is a dose of about 2700 SU, and the third weekly subcutaneous injection is a dose of about 6000 SU.

In some embodiments, the build-up phase and maintenance phase are separated by about 1 month. In some embodiments, the build-up phase and maintenance phase are separated by about 21 to about 35 days.

In some embodiments, the maintenance phase consists of: (i) a first monthly subcutaneous injection of a composition comprising one or more antigens in a dose of about 5000 to about 8000 SU; (ii) a second monthly subcutaneous injection of a composition comprising one or more antigens in a dose of about 5000 to about 8000 SU; and (iii) a third monthly subcutaneous injection of a composition comprising one or more antigens in a dose of about 5000 to about 8000 SU. In some embodiments, the second monthly subcutaneous injection is administered about 21 to about 35 days after the first subcutaneous injection and the third monthly subcutaneous injection is administered about 21 to about 35 days after the second monthly subcutaneous injection. In some embodiments, the first monthly subcutaneous injection is a dose of about 6000 SU, the second monthly subcutaneous injection is a dose of about 6000 SU, and the third monthly subcutaneous injection is a dose of about 6000 SU.

In one aspect, the present invention provides a method of treating allergic rhinitis and/or rhinoconjunctivitis in a subject, the method comprising administering to a subject in need thereof a therapeutically effective allergen-specific immunotherapy comprising or consisting of: (i) a first subcutaneous injection of a composition comprising one or more antigens in a dose of about 300 to about 1500 SU; (ii) a second subcutaneous injection of a composition comprising one or more antigens in a dose of about 2000 to about 4000 SU, wherein the second subcutaneous injection is administered about 6 to about 9 days after the first subcutaneous injection; (iii) a third subcutaneous injection of a composition comprising one or more antigens in a dose of about 5000 to about 8000 SU, wherein the third subcutaneous injection is administered about 6 to about 9 days after the second subcutaneous injection; (iv) a fourth subcutaneous injection of a composition comprising one or more antigens in a dose of about 5000 to about 8000 SU, wherein the fourth subcutaneous injection is administered about 21 to about 35 days after the third subcutaneous injection; (v) a fifth subcutaneous injection of a composition comprising one or more antigens in a dose of about 5000 to about 8000 SU, wherein the fifth subcutaneous injection is administered about 21 to about 35 days after the fourth subcutaneous injection; and (vi) a sixth subcutaneous injection of a composition comprising one or more antigens in a dose of about 5000 to about 8000 SU, wherein the sixth subcutaneous injection is administered about 21 to about 35 days after the fifth subcutaneous injection.

In some embodiments, the first subcutaneous injection is a dose of about 900 SU, the second subcutaneous injection is a dose of about 2700 SU, the third subcutaneous injection is a dose of about 6000 SU, the fourth subcutaneous injection is a dose of about 6000 SU, the fifth subcutaneous injection is a dose of about 6000 SU, and the sixth subcutaneous injection is a dose of about 6000 SU.

In some embodiments, the final subcutaneous injection is administered within about 2 months prior to the predicted start of the allergy season. In some embodiments, the final subcutaneous injection is administered within about 1 month to about 2 months prior to the predicted start of the allergy season. In some embodiments, the final subcutaneous injection is administered within about 1 week to about 7 weeks prior to the predicted start of the allergy season.

Each subcutaneous injection may be administered in any suitable volume and by any suitable mode of administration. In some embodiments, each subcutaneous injection is about 0.5 mL to about 1.0 mL. In some embodiments, each subcutaneous injection is to the lateral/posterior aspect of the middle third of the upper arm. In some embodiments, the subcutaneous injection sites are alternated between arms.

The method of the present invention may be used to treat any suitable subject in need thereof. In some embodiments, the rhinitis and/or rhinoconjunctivitis is caused by an IgE mediated allergy. In some embodiments, the rhinitis and/or rhinoconjunctivitis is caused by an IgE mediated allergy against grass/rye, tree and/or weed pollen. In some embodiments, the subject is a human. In some embodiments, the subject is an adult, an adolescent or a child over the age of five. In some embodiments, the subject has rhinitis and/or rhinoconjunctivitis caused by an IgE mediated allergy. In some embodiments, the subject has rhinitis and/or rhinoconjunctivitis caused by an IgE mediated allergy against grass/rye, tree or weed pollen. In some embodiments, the subject has been diagnosed with a positive skin prick test and/or specific IgE test.

In one aspect, the present invention provides a kit for treating allergic rhinitis and/or rhinoconjunctivitis in a subject, wherein the kit comprises: (i) a first vial comprising about 0.5 to about 2.0 mL of a composition comprising one or more antigens in a dose of about 300 to about 1500 SU/mL; (ii) a second vial comprising about 0.5 to about 2.0 mL of a composition comprising one or more antigens in a dose of about 2000 to about 4000 SU/mL; (iii) a third vial comprising about 0.5 to about 2.0 mL of a composition comprising one or more antigens in a dose of about 5000 to about 8000 SU/mL; (iv) a fourth vial comprising about 0.5 to about 2.0 mL of a composition comprising one or more antigens in a dose of about 5000 to about 8000 SU/mL; (v) a fifth vial comprising about 0.5 to about 2.0 mL of a composition comprising one or more antigens in a dose of about 5000 to about 8000 SU/mL; and (vi) a sixth vial comprising about 0.5 to about 2.0 mL of a composition comprising one or more antigens in a dose of about 5000 to about 8000 SU/mL. In some embodiments, the first vial comprises a dose of about 900 SU/mL, the second vial comprises a dose of about 2700 SU/mL, the third vial comprises a dose of about 6000 SU/mL, the fourth vial comprises a dose of about 6000 SU/mL, the fifth vial comprises a dose of about 6000 SU/mL, and the sixth vial comprises a dose of about 6000 SU/mL. In some embodiments, the kit comprises instructions for a method of treating allergic rhinitis and/or rhinoconjunctivitis in a subject.

In one aspect, the present invention provides a kit for treating allergic rhinitis and/or rhinoconjunctivitis in a subject, wherein the kit comprises: (i) a first syringe comprising about to about 1.0 mL of a composition comprising one or more antigens in a dose of about 300 to about 1500 SU/mL; (ii) a second syringe comprising about 0.5 mL to about 1.0 mL of a composition comprising one or more antigens in a dose of about 2000 to about 4000 SU/mL; (iii) a third syringe comprising about 0.5 mL to about 1.0 mL of a composition comprising one or more antigens in a dose of about 5000 to about 8000 SU/mL; (iv) a fourth syringe comprising about 0.5 mL to about 1.0 mL of a composition comprising one or more antigens in a dose of about 5000 to about 8000 SU/mL; (v) a fifth syringe comprising about 0.5 mL to about 1.0 mL of a composition comprising one or more antigens in a dose of about 5000 to about 8000 SU/mL; and (vi) a sixth syringe comprising about 0.5 mL to about 1.0 mL of a composition comprising one or more antigens in a dose of about 5000 to about 8000 SU/mL. In some embodiments, the first syringe comprises a dose of about 900 SU/mL, the second syringe comprises a dose of about 2700 SU/mL, the third syringe comprises a dose of about 6000 SU/mL, the fourth syringe comprises a dose of about 6000 SU/mL, the fifth syringe comprises a dose of about 6000 SU/mL, and the sixth syringe comprises a dose of about 6000 SU/mL. In some embodiments, the kit comprises instructions for a method of treating allergic rhinitis and/or rhinoconjunctivitis in a subject.

The one or more antigens may be any suitable antigens. In some embodiments, the one or more antigens are derived from pollen. In some embodiments, the one or more antigens are derived from Bent grass pollen, Foxtail pollen, Sweet vernal pollen, False oat pollen, Brome pollen, Dogstail pollen, Cocksfoot pollen, Fescue pollen, Yorkshire Fog pollen, Rye grass pollen, Timothy grass pollen, Meadow grass pollen and Cultivated Rye pollen. In some embodiments, the one or more antigens are extracted from an antigen-containing material. In some embodiments, the one or more antigens are diafiltered using a membrane with a 5 to 10 kDa molecular weight cut-off. In some embodiments, the one or more antigens are modified with glutaraldehyde. In some embodiments, the one or more antigens are adsorbed onto L-tyrosine.

Each composition may comprise any suitable purified allergen extract. In some embodiments, each composition comprising one or more antigens comprises purified allergen extracts of pollen. In some embodiments, each composition comprising one or more antigens comprises equal amounts of purified allergen extracts of pollen from the following grasses: Bent grass, Foxtail, Sweet vernal, False oat, Brome, Dogstail, Cocksfoot, Fescue, Yorkshire Fog, Rye grass, Timothy grass, Meadow grass and Cultivated Rye. In some embodiments, the allergens are modified into allergoids by treatment with glutaraldehyde. In some embodiments, the allergens are adsorbed onto L-tyrosine.

Each composition may comprise any suitable adsorbant base in any suitable amount. In some embodiments, each composition comprising one or more antigens comprises an amino acid. In some embodiments, the amino acid is tyrosine, tryptophan or a derivative thereof. In some embodiments, the amino acid is L-tyrosine. In some embodiments, the L-tyrosine is in the form of microcrystalline tyrosine (MCT). In some embodiments, the MCT has a median particle length of about 10 to about 100 microns, optionally about 10 to about microns; and/or the MCT has a median particle width of about 1 to about 10 microns, optionally about 2 to about 6 microns. In some embodiments, each composition comprising one or more antigens comprises the adsorbant base in an amount of about 0.5% w/v to about 5% w/v, optionally about 2% w/v or 3% w/v.

Each composition may comprise any suitable adjuvant in any suitable amount. In some embodiments, each composition comprising one or more antigens comprises an adjuvant. In some embodiments, the adjuvant is MPL, 3-DMPL or a derivative or salt thereof. In some embodiments, the MPL, 3-DMPL or a derivative or salt thereof has a mean particle size of about 500 nm or less or about 200 nm or less. In some embodiments, each composition comprising one or more antigens comprises the adjuvant in an amount of about 25 μg/mL to about 100 μg/mL, optionally about 50 μg/mL.

In some embodiments, each composition comprising one or more antigens comprises L-tyrosine in an amount of about 2% w/v and MPL, 3-DMPL or a derivative or salt thereof in an amount of about 50 μg/mL.

In some embodiments, each composition comprising one or more antigens further comprises dipalmitoyl phosphatidylcholine (DPPC), phenol, sodium chloride, disodium phosphate dodecahydrate, sodium dihydrogen phosphate dihydrate and water. In some embodiments, each composition comprising one or more antigens comprises DPPC in an amount of about to about 0.01 mg/ml, optionally about 0.006 mg/ml. In some embodiments, each composition comprising one or more antigens comprises phenol in an amount of amount 1 to about 10 mg/ml, optionally about 5 mg/ml. In some embodiments, each composition comprising one or more antigens comprises sodium chloride in an amount of from about 5 to about 20 mg/mL, optionally about 9.0 mg/mL. In some embodiments, each composition comprising one or more antigens comprises disodium phosphate dodecahydrate and sodium dihydrogen phosphate dihydrate in an amount of about 1 to about 10 mg/mL, optionally about 4.0 mg/mL. In some embodiments, each composition comprising one or more antigens is about pH 7.

### BRIEF DESCRIPTION OF THE DRAWING

FIGS. 1A and 1B—Schematic Showing Conventional and Extended Posology

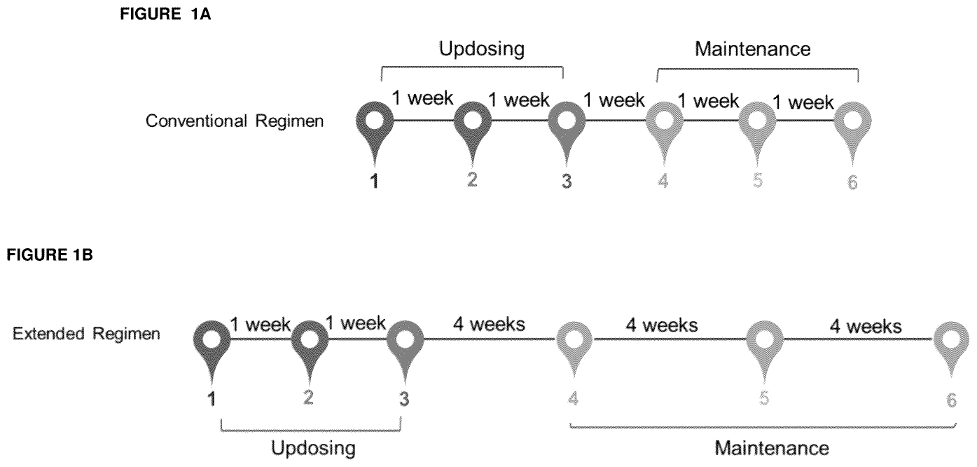

Schematic showing (1A) conventional posology (6 weekly injections); and (1B) extended posology (3 weekly injections followed by 3 monthly injections).

FIG. 2—Schematic Showing Exploratory Field Study Design

An exploratory field study was carried out to explore the efficacy and safety of a subcutaneous immunotherapy in subjects with seasonal allergic rhinitis and/or rhinoconjunctivitis induced by grass pollen exposure.

FIG. 3—Schematic Showing Combined Symptom Medication Score (CSMS)

The combined symptom medication score (CSMS) was calculated from the daily symptom score (dSS) and daily medication score (dMS). The dSS for CSMS was calculated as the sum of the scores for the 6 individual symptoms assessed in a 4 point severity scale (0-No symptoms to 3-Severe symptoms), divided by 6 (i.e., ranging from 0 to 3). The dMS of the CSMS was assessed from a score 0 (no relief medication) to 3 (highest step relief medication) per day.

FIGS. 4A-4C—Primary Efficacy Analysis (CSMS)

Analysis of CSMS during peak grass pollen season: (4A) mean CSMS during peak grass pollen season for conventional and extended posology and placebo groups; (4B) absolute change from placebo during peak pollen season for conventional and extended posology; (4C) percentage change from placebo during peak pollen season for conventional and extended posology. *p<0.05

FIG. 5—Post Hoc Sensitivity Analyses of Primary Endpoint (CSMS)

Absolute CSMS change during peak grass pollen season compared to placebo, corrected for environmental data.

FIGS. 6A-6C— Secondary Efficacy Analyses (CSMS and TCS)

For conventional and extended posology: (6A) CSMS change from placebo over entire grass pollen season; (6B) TCS change from placebo over entire grass pollen season; and (6C) TCS change from placebo over peak grass pollen season. *p<0.05, **0.05≤p<0.10, ***0.10≤p<0.25.

FIGS. 7A-7D—Secondary Efficacy Analyses (dSS, dMS, Well and Severe Days)

For conventional and extended posology: (7A) dSS change from placebo over peak grass pollen season; (7B) dMS change from placebo over peak grass pollen season; (7C) increase in Well days odds ratio from placebo over peak grass pollen season; and (7D) reduction in Severe days odds ratio from placebo over peak grass pollen season. *p<0.05, **0.05≤p<0.10, ***0.10≤p<0.25, ****p>0.25.

FIGS. 8A-8C—Secondary Efficacy Analyses (RQLQ)

For conventional and extended posology: (8A) RQLQ change from placebo over peak grass pollen season, observed cases; (8B) RQLQ change from placebo over peak grass pollen season, per protocol; and (8C) RQLQ change from placebo over entire grass pollen season. *p<0.05, **0.05 p<0.10, ***0.10 p<0.25, ****p>0.25.

FIGS. 9A-9B— Secondary Efficacy Analyses (Serum IgG4)

For conventional and extended posology: (9A) Serum IgG4 change from placebo during grass pollen season, observed cases; and (9B) Serum IgG4 change from placebo during grass pollen season, per protocol. *p<0.05.

FIG. 10A-10B— Summary of Efficacy Analyses

(10A) Summary of eDiary endpoints during the peak and entire GPS for the extended and conventional regimen groups compared to placebo (mean±80% CI). CSMS, combined symptom and medication score; GPS, grass pollen season; TCS, total combined score.

(10B) Change from baseline in RQLQ-S(A) and grass-specific IgG4 (B) after PQ Grass compared to placebo. Error bars represent standard error of the mean; extended regimen group n=40, placebo group n=20. IgG4, immunoglobulin G4; RQLQ-S, Rhinoconjunctivitis Quality Of Life Questionnaire standardized.

FIGS. 11A-11C—Biomarker Analyses (Nasal Grass-Specific IgA2, IgG and IgG4)

For conventional and extended posology and placebo groups. Nasal grass-specific IgA2, IgG and IgG4 was determined at baseline, pre-grass pollen season (Pre-GPS), and post-grass pollen season (Post-GPS): (11A) grass-specific nasal IgA2; (11B) grass-specific nasal IgG; and (11C) grass-specific nasal IgG4.

FIGS. 12A-12C—Biomarker Analyses (Th2 and Th2A)

For conventional and extended posology and placebo groups. Proliferated Th2 and Th2A cells was determined at baseline, pre-grass pollen season (Pre-GPS), and post-grass pollen season (Post-GPS): (12A) change in proliferated Th2 cells; and (12B) change in proliferated Th2A cells. (12C) FIowSOM analysis confirm reduced meta-cluster corresponding to Th2/Th2A in both conventional and Extended AIT compared to Placebo.

FIGS. 13A-13C—Biomarker Analyses (Tfh Cells)

For conventional and extended posology and placebo groups. Proliferated T follicular helper (Tfh) cells, IL-10⁺ Tfh cells, and IFNγ⁺ Tfh cells was determined at baseline, pre-grass pollen season (Pre-GPS), and post-grass pollen season (Post-GPS): (13A) change in proliferated Tfh cells, (13B) change in IL-10⁺ Tfh cells; and (13C) changes in IFNγ⁺ Tfh cells.

### DETAILED DESCRIPTION

Various preferred features and embodiments of the present invention will now be described by way of non-limiting examples. This disclosure is not limited by the exemplary methods and materials disclosed herein, and any methods and materials similar or equivalent to those described herein can be used in the practice or testing of embodiments of this disclosure. The skilled person will understand that they can combine all features of the invention disclosed herein without departing from the scope of the invention as disclosed.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes”, “containing”, or “contains”, and are inclusive or open-ended and do not exclude additional, non-recited members, elements or steps. The terms “comprising”, “comprises” and “comprised of” also include the term “consisting of”.

Numeric ranges are inclusive of the numbers defining the range. As used herein the term “about” means approximately, in the region of, roughly, or around. When the term “about” is used in conjunction with a numerical value or range, it modifies that value or range by extending the boundaries above and below the numerical value(s) set forth. In general, the terms “about” and “approximately” may be used herein to modify a numerical value(s) above and below the stated value(s) by 10%.

The publications discussed herein are provided solely for their disclosure prior to the filing date of the present application. Nothing herein is to be construed as an admission that such publications constitute prior art to the claims appended hereto.

The practice of the present invention will employ, unless otherwise indicated, conventional techniques of which are within the capabilities of a person of ordinary skill in the art. Such techniques are explained in the literature. All publications mentioned in the specification are herein incorporated by reference.

Methods of Treatment

The present invention provides a method of treating allergic rhinitis and/or rhinoconjunctivitis in a subject, the method comprising administering to a subject in need thereof a therapeutically effective allergen-specific immunotherapy.

As used herein, an “allergen-specific immunotherapy” (also known as “allergen immunotherapy”, “specific immunotherapy”, “desensitization”, “hypo-sensitization”, or “allergy shots”) may refer to a medical treatment for environmental allergies involving exposing a subject to increased doses of allergen.

The allergen-specific immunotherapy of the present invention may be a subcutaneous allergen-specific immunotherapy. As used herein, a “subcutaneous allergen-specific immunotherapy” or “SCIT” may refer to an allergen-specific immunotherapy where the doses of allergen are administered by subcutaneous injection.

The allergen-specific immunotherapy of the present invention may comprise or consist of: (a) a build-up phase; and (b) a maintenance phase. As used herein, a “build-up phase” (also known as “up-dosing” or “induction”) may refer to a period in which a subject is administered a gradually increasing dose of allergen until a dose is reached that is effective in inducing immunologic tolerance to the allergen. As used herein, a “maintenance phase” may refer to the period after which a subject has reached a dose that is effective in inducing immunologic tolerance to an allergen and during which the subject may be administered a constant dose of allergen.

Dosage Schedule

The build-up phase may comprise of consist of three weekly subcutaneous injections of a composition comprising one or more antigens. In some embodiments, the second weekly subcutaneous injection is administered about 1 week after the first subcutaneous injection. In some embodiments, the third weekly subcutaneous injection is administered about 1 week after the second subcutaneous injection. In some embodiments, the second weekly subcutaneous injection is administered about 6 to about 9 days (e.g. about 6 days, about 7 days, about 8 days, or about 9 days) after the first subcutaneous injection. In some embodiments, the third weekly subcutaneous injection is administered about 6 to about 9 days (e.g. about 6 days, about 7 days, about 8 days, or about 9 days) after the second subcutaneous injection.

The build-up phase and maintenance phase may be separated by any suitable duration. In some embodiments, the build-up phase and maintenance phase are separated by about 1 month. In some embodiments, the build-up phase and maintenance phase are separated by about 21 to about 35 days (e.g. about 21 days, about 22 days, about 23 days, about 24 days, about 25 days, about 26 days, about 27 days, about 28 days, about 29 days, about 30 days, about 31 days, about 32 days, about 33 days, about 34 days, or about 35 days).

The maintenance phase may comprise of consist of three monthly subcutaneous injections of a composition comprising one or more antigens. In some embodiments, the second monthly subcutaneous injection is administered about 1 month after the first subcutaneous injection. In some embodiments, the third monthly subcutaneous injection is administered about 1 month after the second monthly subcutaneous injection. In some embodiments, the second monthly subcutaneous injection is administered about 21 to about 35 days (e.g. about 21 days, about 22 days, about 23 days, about 24 days, about 25 days, about 26 days, about 27 days, about 28 days, about 29 days, about 30 days, about 31 days, about 32 days, about 33 days, about 34 days, or about 35 days) after the first subcutaneous injection. In some embodiments, the third monthly subcutaneous injection is administered about 21 to about 35 days (e.g. about 21 days, about 22 days, about 23 days, about 24 days, about 25 days, about 26 days, about 27 days, about 28 days, about 29 days, about 30 days, about 31 days, about 32 days, about 33 days, about 34 days, or about 35 days) after the second monthly subcutaneous injection.

The maintenance phase may comprise one or more further monthly subcutaneous injections. For example, the maintenance phase may comprise one, two, three, or four or more further monthly subcutaneous injections of a composition comprising one or more antigens. Each monthly subcutaneous injection may be administered about 1 month after the preceding monthly subcutaneous injection. Each monthly subcutaneous injection may be administered about 21 to about 35 days (e.g. about 21 days, about 22 days, about 23 days, about 24 days, about 25 days, about 26 days, about 27 days, about 28 days, about 29 days, about 30 days, about 31 days, about 32 days, about 33 days, about 34 days, or about 35 days) after the preceding monthly subcutaneous injection.

The final subcutaneous injection may be administered within about 2 months prior to the predicted start of the allergy season. For example, if the one or more antigens are pollen antigens, the final subcutaneous injection may be administered within about 2 months prior to the predicted start of the pollen season. As used herein, a “pollen season” may refer to the period with a defined amount of pollen in the ambient air (see e.g. Bastl, K., et al., 2018. Current Allergy and Asthma Reports, 18(12), pp. 1-10). In some embodiments, the pollen season is the EAACI pollen season (see e.g. Pfaar, O., et al., 2017. Allergy, 72(5), pp. 713-722).

In some embodiments, the final subcutaneous injection is administered within about 1 month to about 2 months prior to the predicted start of the allergy season. In some embodiments, the final subcutaneous injection is administered within about 7 weeks prior to the predicted start of the allergy season. In some embodiments, the final subcutaneous injection is administered within about 1 week to about 7 weeks prior to the predicted start of the allergy season. In some embodiments, the final subcutaneous injection is administered within about 3 weeks to about 7 weeks prior to the predicted start of the allergy season. In some embodiments, the final subcutaneous injection is administered within about 4 weeks to about 7 weeks prior to the predicted start of the allergy season. In some embodiments, the final subcutaneous injection is administered within about 49 days prior to the predicted start of the allergy season. In some embodiments, the final subcutaneous injection is administered within about 7 days to about 49 days prior to the predicted start of the allergy season. In some embodiments, the final subcutaneous injection is administered within about 21 days to about 49 days prior to the predicted start of the allergy season. In some embodiments, the final subcutaneous injection is administered within about 28 days to about 49 days prior to the predicted start of the allergy season.

Dosage Quantities

The dose of allergen administered in each subcutaneous injection may be any suitable dose. The dosage regimen may, at least in part, be determined by the need of the individual subject and be dependent upon the judgement of the medical practitioner.

Typically, manufacturers use several types of allergen units: histamine equivalent in prick testing (HEP), biologic or diagnostic unit (BU or DU), bioequivalent allergen unit (BAU), Therapeutic Unit (TU) or Standardized Unit (SU). However, regardless of the unitage employed, manufacturers may correlate in vivo standardization with in vitro testing using different factors. Once overall strength of the original extract is determined by using an in vivo method, this data is used to develop an in-house reference preparation (IHRP) to estimate the strength of extracts from other batches by an in vitro method. The IHRP can then be cross validated over time as the materials reach their expiry. ELISA is commonly applied as a measure of in vitro biological activity.

Correlation of in vivo biological standardization, by appropriate methods on the basis of skin reactivity test using methods (e.g. such as those described by Turkeltaub, P. C., 1989. Allergologia et Immunopathologia, 17(2), pp. 53-65 and Nordic Council of Medicines. Registration of Allergenic Preparations. Nordic Guidelines, Vol. 23, 2nd edition. Uppsala, Sweden: NLN Publications 1988. pp 1-34) and corresponding in vitro allergenic activity of the (first) IHRP should be performed, with potency labelling based upon in vitro testing (see e.g. EMEA/CHMP/BWP/304831/2007). Each new IHRP may then prepared and compared against the previously assigned preparation, and thus can provide an unbroken link back to original in vivo biological standardization studies.

In the build-up phase, each subcutaneous injection may have an increased dose of allergen, until a dose is reached that is effective in inducing immunologic tolerance to the allergen.

In some embodiments, the first weekly subcutaneous injection is a dose of about 300 to about 1500 SU (e.g. about 300 SU, about 400 SU, about 500 SU, about 600 SU, about 700 SU, about 800 SU, about 900 SU, about 1000 SU, about 1100 SU, about 1200 SU, about 1300 SU, about 1400 SU, or about 1500 SU). In some embodiments, the first weekly subcutaneous injection is a dose of about 900 SU.

In some embodiments, the second weekly subcutaneous injection is a dose of about 2000 to about 4000 SU (e.g. about 2000 SU, about 2100 SU, about 2200 SU, about 2300 SU, about 2400 SU, about 2500 SU, about 2600 SU, about 2700 SU, about 2800 SU, about 2900 SU, about 3000 SU, about 3100 SU, about 3200 SU, about 3300 SU, about 3400 SU, about 3500 SU, about 3600 SU, about 3700 SU, about 3800 SU, about 3900 SU, or about 4000 SU). In some embodiments, the second weekly subcutaneous injection is a dose of about 2700 SU.

In some embodiments, the third weekly subcutaneous injection is a dose of about 5000 to about 8000 SU (e.g. about 5000 SU, about 5200 SU, about 5400 SU, about 5600 SU, about 5800 SU, about 6000 SU, about 6200 SU, about 6400 SU, about 6600 SU, about 6800 SU, about 7000 SU, about 7200 SU, about 7400 SU, about 7600 SU, about 7800 SU, or about 8000 SU). In some embodiments, the third weekly subcutaneous injection is a dose of about 6000 SU.

In the maintenance phase, each subcutaneous injection may have a constant dose of allergen that is effective in inducing immunologic tolerance to the allergen.

In some embodiments, the first monthly subcutaneous injection is a dose of about 5000 to about 8000 SU (e.g. about 5000 SU, about 5200 SU, about 5400 SU, about 5600 SU, about 5800 SU, about 6000 SU, about 6200 SU, about 6400 SU, about 6600 SU, about 6800 SU, about 7000 SU, about 7200 SU, about 7400 SU, about 7600 SU, about 7800 SU, or about 8000 SU). In some embodiments, the first monthly subcutaneous injection is a dose of about 6000 SU.

In some embodiments, the second monthly subcutaneous injection is a dose of about 5000 to about 8000 SU (e.g. about 5000 SU, about 5200 SU, about 5400 SU, about 5600 SU, about 5800 SU, about 6000 SU, about 6200 SU, about 6400 SU, about 6600 SU, about 6800 SU, about 7000 SU, about 7200 SU, about 7400 SU, about 7600 SU, about 7800 SU, or about 8000 SU). In some embodiments, the second monthly subcutaneous injection is a dose of about 6000 SU.

In some embodiments, the third monthly subcutaneous injection is a dose of about 5000 to about 8000 SU (e.g. about 5000 SU, about 5200 SU, about 5400 SU, about 5600 SU, about 5800 SU, about 6000 SU, about 6200 SU, about 6400 SU, about 6600 SU, about 6800 SU, about 7000 SU, about 7200 SU, about 7400 SU, about 7600 SU, about 7800 SU, or about 8000 SU). In some embodiments, the third monthly subcutaneous injection is a dose of about 6000 SU.

In some embodiments, each monthly subcutaneous injection is a dose of about 5000 to about 8000 SU (e.g. about 5000 SU, about 5200 SU, about 5400 SU, about 5600 SU, about 5800 SU, about 6000 SU, about 6200 SU, about 6400 SU, about 6600 SU, about 6800 SU, about 7000 SU, about 7200 SU, about 7400 SU, about 7600 SU, about 7800 SU, or about 8000 SU). In some embodiments, each monthly subcutaneous injection is a dose of about 6000 SU.

SU can correlate to total protein content of modified grass measured in the terms of Protein Nitrogen Units (PNU) and a conversion factor may be readily determined by one of skill in the art. Typically, 1 SU may be about 0.6 to about 1.0 PNU. In some embodiments, 1 SU may be about 0.8 PNU. For example: a dose of about 300 SU to about 1500 SU may correspond to about 240 PNU to about 1200 PNU; a dose of about 2000 SU to about 4000 SU may correspond to about 1600 PNU to about 3200 PNU; and a dose of about 5000 SU to about 8000 SU may correspond to about 4000 PNU to about 6400 PNU. For example: a dose of about 900 SU may correspond to about 720 PNU; a dose of about 2700 may correspond to about 2160 PNU; and a dose of about 6000 SU may correspond to about 4800 PNU.

Exemplary Dosage Regimens

In some embodiments, the allergen-specific immunotherapy of the present invention comprises or consists of: (i) a first subcutaneous injection of a composition comprising one or more antigens in a dose of about 300 to about 1500 SU; (ii) a second subcutaneous injection of a composition comprising one or more antigens in a dose of about 2000 to about 4000 SU, wherein the second subcutaneous injection is administered about 1 week after the first subcutaneous injection; (iii) a third subcutaneous injection of a composition comprising one or more antigens in a dose of about 5000 to about 8000 SU, wherein the third subcutaneous injection is administered about 1 week after the second subcutaneous injection; (iv) a fourth subcutaneous injection of a composition comprising one or more antigens in a dose of about 5000 to about 8000 SU, wherein the fourth subcutaneous injection is administered about 1 month after the third subcutaneous injection; (v) a fifth subcutaneous injection of a composition comprising one or more antigens in a dose of about 5000 to about 8000 SU, wherein the fifth subcutaneous injection is administered about 1 month after the fourth subcutaneous injection; and (vi) a sixth subcutaneous injection of a composition comprising one or more antigens in a dose of about 5000 to about 8000 SU, wherein the sixth subcutaneous injection is administered about 1 month after the fifth subcutaneous injection.

In some embodiments, the allergen-specific immunotherapy of the present invention comprises or consists of: (i) a first subcutaneous injection of a composition comprising one or more antigens in a dose of about 300 to about 1500 SU; (ii) a second subcutaneous injection of a composition comprising one or more antigens in a dose of about 2000 to about 4000 SU, wherein the second subcutaneous injection is administered about 6 to about 9 days after the first subcutaneous injection; (iii) a third subcutaneous injection of a composition comprising one or more antigens in a dose of about 5000 to about 8000 SU, wherein the third subcutaneous injection is administered about 6 to about 9 days after the second subcutaneous injection; (iv) a fourth subcutaneous injection of a composition comprising one or more antigens in a dose of about 5000 to about 8000 SU, wherein the fourth subcutaneous injection is administered about 21 to about 35 days after the third subcutaneous injection; (v) a fifth subcutaneous injection of a composition comprising one or more antigens in a dose of about 5000 to about 8000 SU, wherein the fifth subcutaneous injection is administered about 21 to about 35 days after the fourth subcutaneous injection; and (vi) a sixth subcutaneous injection of a composition comprising one or more antigens in a dose of about 5000 to about 8000 SU, wherein the sixth subcutaneous injection is administered about 21 to about 35 days after the fifth subcutaneous injection.

In some embodiments, the first subcutaneous injection is a dose of about 900 SU, the second subcutaneous injection is a dose of about 2700 SU, the third subcutaneous injection is a dose of about 6000 SU, the fourth subcutaneous injection is a dose of about 6000 SU, the fifth subcutaneous injection is a dose of about 6000 SU, and the sixth subcutaneous injection is a dose of about 6000 SU.

Mode of Administration

Each subcutaneous injection may be administered in any suitable volume and by any suitable mode of administration.

In some embodiments, the subcutaneous injection volume is from about 0.5 mL to about 2.0 mL (e.g. about 0.5 mL, about 0.6 mL, about 0.7 mL, about 0.8 mL, about 0.9 mL, about 1.0 mL, about 1.1 mL, about 1.2 mL, about 1.3 mL, about 1.4 mL, about 1.5 mL, about 1.6 mL, about 1.7 mL, about 1.8 mL, about 1.9 mL, or about 2.0 mL). In some embodiments, the subcutaneous injection volume is about 1.0 mL.

In some embodiments, prior to subcutaneous injection the solution is brought to room temperature. In some embodiments, the suspension for subcutaneous injection is kept at room temperature for about 2 hours before injection or warmed in the hand.

In some embodiments, each subcutaneous injection is administered to the upper arm. In some embodiments, each subcutaneous injection is administered to the lateral or posterior aspect of the middle third of the upper arm. In some embodiments, the subcutaneous injection sites are alternated between arms (for example, if the first subcutaneous injection is in the subject's right arm, the second subcutaneous injection is in the subject's left arm) or differing sites on one arm. In some embodiments, repeated injections at the same injection site are avoided.

Compositions Comprising One or More Antigens

The allergen-specific immunotherapy of the present invention comprises a plurality of subcutaneous injections of a composition comprising one or more antigens.

In some embodiments, each composition is identical, except for the dose of one or more antigens. For example, each composition may comprise the same antigens in the same relative quantities (albeit in a different total dose) and the same carriers, diluents, and excipients. In other embodiments, one or more of the compositions may differ in respects other than the dose of one or more antigens.

In some embodiments, each composition comprising one or more antigens is a composition according to the present invention (as described in the section entitled “Compositions”).

Disease to be Treated

The present invention provides a method of treating an allergy. In some embodiments, the present invention provides a method of treating allergic rhinitis, allergic conjunctivitis, and/or allergic rhinoconjunctivitis in a subject.

As used herein, an “allergy” or “allergic disease” may refer to a condition caused by hypersensitivity of the immune system to typically harmless substances in the environment. Allergic diseases may include allergic rhinitis, allergic conjunctivitis, allergic rhinoconjunctivitis, food allergies, atopic dermatitis, allergic asthma, and anaphylaxis.

As used herein, “allergic rhinitis” may refer to an inflammatory disorder of nasal mucosa, which is characterized by pruritus, sneezing, rhinorrhea, and nasal congestion that is mediated by an allergic response (see e.g. Greiner, A. N., et al., 2011. The Lancet, 378(9809), pp. 2112-2122). Allergic rhinitis is also often accompanied by allergic conjunctivitis.

As used herein, “allergic conjunctivitis” may refer to an inflammatory disorder of the ocular surface which is characterized by itching, tearing, lid and conjunctival edema—redness that is mediated by an allergic response (see e.g. Ono, S. J. and Abelson, M. B., 2005. Journal of Allergy and Clinical Immunology, 115(1), pp. 118-122).

As used herein, “allergic rhinoconjunctivitis” may refer to an inflammatory disorder of the nose and eyes, resulting in a chronic, mostly eosinophilic, inflammation of the nasal mucosa and conjunctiva that is mediated by an allergic response. Symptoms include itching, sneezing, watery nasal discharge, and nasal congestion. Commonly, there are associated ocular symptoms such as watery, red and/or itchy eyes (see e.g. Roberts, G., et al., 2018. Allergy, 73(4), pp. 765-798).

In some embodiments, the allergic rhinitis, allergic conjunctivitis, and/or allergic rhinoconjunctivitis is caused by an IgE mediated allergy. In some embodiments, the allergic rhinitis, allergic conjunctivitis, and/or allergic rhinoconjunctivitis is caused by an IgE mediated allergy against pollen. In some embodiments, the allergic rhinitis, allergic conjunctivitis, and/or allergic rhinoconjunctivitis is caused by an IgE mediated allergy against grass/rye, tree or weed pollen. In some embodiments, the allergic rhinitis, allergic conjunctivitis, and/or allergic rhinoconjunctivitis is hay fever or seasonal allergic rhinitis, allergic conjunctivitis, and/or allergic rhinoconjunctivitis induced by grass/rye, tree or weed pollen exposure.

In some embodiments, the allergic rhinitis, allergic conjunctivitis, and/or allergic rhinoconjunctivitis is caused by an IgE mediated allergy against grass/rye pollen, such as Bent grass pollen, Foxtail pollen, Sweet vernal pollen, False oat pollen, Brome pollen, Dogstail pollen, Cocksfoot pollen, Fescue pollen, Yorkshire Fog pollen, Rye grass pollen, Timothy grass pollen, Meadow grass pollen, or Cultivated Rye pollen. In some embodiments, the allergic rhinitis, allergic conjunctivitis, and/or allergic rhinoconjunctivitis is caused by an IgE mediated allergy against weed pollen, such as Ragweed pollen. In some embodiments, the allergic rhinitis, allergic conjunctivitis, and/or allergic rhinoconjunctivitis is caused by an IgE mediated allergy against tree pollen, such as Birch pollen, Alder pollen, or Hazel pollen. The allergic rhinitis, allergic conjunctivitis, and/or allergic rhinoconjunctivitis may be seasonal and induced by the pollen exposure.

The method of the present invention may lessen, reduce, or improve at least one symptom of allergic rhinitis, allergic conjunctivitis, and/or allergic rhinoconjunctivitis. The symptom assessment may be based on the recommendations of the EAACI (see Pfaar, O., et al., 2014. Allergy, 69(7), pp. 854-867). In some embodiments, the method of the present invention may lessen, reduce, or improve at least one symptom selected from itchy/red eyes, watery eyes, itchy nose, blocked nose, runny nose, itchy nose, and sneezing. In some embodiments, the method of the present invention may reduce the subject's daily symptom score (dSS), which may, for example, be calculated as described herein.

The method of the present invention may reduce the amount of or eliminate relief medication used by the subject. The medication assessment may be based on the recommendations of EAACI (see Pfaar, O., et al., 2014. Allergy, 69(7), pp. 854-867). In some embodiments, the method of the present invention may reduce the amount of or eliminate the use of oral antihistamine/ocular antihistamine, intranasal corticosteroids, and/or oral corticosteroids by the subject. In some embodiments, the method of the present invention may reduce the subject's daily medication score (dMS), which may, for example, be calculated as described herein.

The method of the present invention may reduce the subject's combined symptom medication score (CSMS). Any suitable CSMS may be used (see e.g. Pfaar, O., et al., 2014. Allergy, 69(7), pp. 854-867). In some embodiments, the CSMS is calculated from the daily symptom score (dSS) and daily medication score (dMS), for example as described herein.

The method of the present invention may reduce the subject's total combined score (TCS). Any suitable TCS may be used. The TCS may be the sum of the daily symptom score (dSS) and daily medication score (dMS), for example as described herein.

The method of the present invention may reduce the subject's rhinoconjunctivitis quality of life questionnaire (RQLQ) score. Any suitable RQLQ may be used, such as a Rhinoconjunctivitis Quality of Life Questionnaire with standardized activities (RQLQ[S]) (see e.g. Juniper, E. F., et al., 1999. Journal of Allergy and Clinical Immunology, 104(2), pp. 364-369).

The method of the present invention may increase the number of well days. A “well day” may be defined as a day with no use of relief medication and a total symptom score of less than or equal to 2 for the 6 rhinitis/rhinoconjunctivitis symptoms (e.g. itchy/red eyes, watery eyes, itchy nose, blocked nose, runny nose, itchy nose, sneezing) and may, for example, be calculated as described herein.

The method of the present invention may reduce the amount of severe days. A “severe day” may be defined as a day with a symptom score of 3 in any of the 6 rhinitis/rhinoconjunctivitis symptoms (e.g. itchy/red eyes, watery eyes, itchy nose, blocked nose, runny nose, itchy nose, sneezing) and may, for example, be calculated as described herein.

The method of the present invention may increase the subject's serum and/or nasal allergen-specific IgG4, allergen-specific IgA2, and/or allergen-specific IgG. In some embodiments, the subject's serum allergen-specific IgG4 is increased by about 2 mg/L, about 2.5 mg/L or about 3 mg/L. In some embodiments, the subject's nasal allergen-specific IgA2 is increased by at least about 10 mgA/mL, about 20 mgA/mL, about 30 mgA/mL, or about 40 mgA/mL. In some embodiments, the subject's nasal allergen-specific IgG is increased by at least about 50 mgA/mL, about 80 mgA/mL, about 100 mgA/mL, or about 150 mgA/mL. In some embodiments, the subject's nasal allergen-specific IgG4 is increased by at least about 20 mgA/mL, about 40 mgA/mL, about 60 mgA/mL, or about 80 mgA/mL.

Subject to be Treated

The method of the present invention may be used to treat any suitable subject in need thereof. In some embodiments, the subject is a mammal. In some embodiments, the subject is a human. In some embodiments, the subject is an adult, an adolescent or a child over the age of five. In some embodiments, the subject is male or female.

In some embodiments, the subject has allergic rhinitis, allergic conjunctivitis, and/or allergic rhinoconjunctivitis caused by an IgE mediated allergy. In some embodiments, the subject has allergic rhinitis, allergic conjunctivitis, and/or allergic rhinoconjunctivitis caused by an IgE mediated allergy against pollen. In some embodiments, the subject has allergic rhinitis, allergic conjunctivitis, and/or allergic rhinoconjunctivitis caused by an IgE mediated allergy against grass/rye, tree or weed pollen. In some embodiments, the subject has hay fever or seasonal allergic rhinitis, allergic conjunctivitis, and/or allergic rhinoconjunctivitis induced by grass/rye, tree or weed pollen exposure.

In some embodiments, the subject has allergic rhinitis, allergic conjunctivitis, and/or allergic rhinoconjunctivitis caused by an IgE mediated allergy against grass/rye pollen, such as Bent grass pollen, Foxtail pollen, Sweet vernal pollen, False oat pollen, Brome pollen, Dogstail pollen, Cocksfoot pollen, Fescue pollen, Yorkshire Fog pollen, Rye grass pollen, Timothy grass pollen, Meadow grass pollen, or Cultivated Rye pollen. In some embodiments, the subject has allergic rhinitis, allergic conjunctivitis, and/or allergic rhinoconjunctivitis caused by an IgE mediated allergy against weed pollen, such as Ragweed pollen. In some embodiments, the subject has allergic rhinitis, allergic conjunctivitis, and/or allergic rhinoconjunctivitis caused by an IgE mediated allergy against tree pollen, such as Birch pollen, Alder pollen, or Hazel pollen. The allergic rhinitis, allergic conjunctivitis, and/or allergic rhinoconjunctivitis may be seasonal and induced by the pollen exposure.

Suitable methods to diagnosis the subject are described in Crobach, M. J., et al., 1998. Scandinavian journal of primary health care, 16(1), pp. 30-36. In some embodiments, the subject has been diagnosed with a positive skin prick test. In some embodiments, the subject has been diagnosed with a specific IgE test.

Kits

The present invention provides a kit for treating allergic rhinitis, allergic conjunctivitis, and/or allergic rhinoconjunctivitis in a subject.

In some embodiments, the kit comprises: (i) a first vial comprising about 0.5 to about 2.0 mL of a composition comprising one or more antigens in a dose of about 300 to about 1500 SU/mL (e.g. about 300 SU, about 400 SU, about 500 SU, about 600 SU, about 700 SU, about 800 SU, about 900 SU, about 1000 SU, about 1100 SU, about 1200 SU, about 1300 SU, about 1400 SU, or about 1500 SU); (ii) a second vial comprising about 0.5 to about 2.0 mL of a composition comprising one or more antigens in a dose of about 2000 to about 4000 SU/mL (e.g. about 2000 SU, about 2100 SU, about 2200 SU, about 2300 SU, about 2400 SU, about 2500 SU, about 2600 SU, about 2700 SU, about 2800 SU, about 2900 SU, about 3000 SU, about 3100 SU, about 3200 SU, about 3300 SU, about 3400 SU, about 3500 SU, about 3600 SU, about 3700 SU, about 3800 SU, about 3900 SU, or about 4000 SU); (iii) a third vial comprising about 0.5 to about 2.0 mL of a composition comprising one or more antigens in a dose of about 5000 to about 8000 SU/mL (e.g. about 5000 SU, about 5200 SU, about 5400 SU, about 5600 SU, about 5800 SU, about 6000 SU, about 6200 SU, about 6400 SU, about 6600 SU, about 6800 SU, about 7000 SU, about 7200 SU, about 7400 SU, about 7600 SU, about 7800 SU, or about 8000 SU); (iv) a fourth vial comprising about 0.5 to about 2.0 mL of a composition comprising one or more antigens in a dose of about 5000 to about 8000 SU/mL (e.g. about 5000 SU, about 5200 SU, about 5400 SU, about 5600 SU, about 5800 SU, about 6000 SU, about 6200 SU, about 6400 SU, about 6600 SU, about 6800 SU, about 7000 SU, about 7200 SU, about 7400 SU, about 7600 SU, about 7800 SU, or about 8000 SU); (v) a fifth vial comprising about 0.5 to about 2.0 mL of a composition comprising one or more antigens in a dose of about 5000 to about 8000 SU/mL (e.g. about 5000 SU, about 5200 SU, about 5400 SU, about 5600 SU, about 5800 SU, about 6000 SU, about 6200 SU, about 6400 SU, about 6600 SU, about 6800 SU, about 7000 SU, about 7200 SU, about 7400 SU, about 7600 SU, about 7800 SU, or about 8000 SU); and (vi) a sixth vial comprising about 0.5 to about 2.0 mL of a composition comprising one or more antigens in a dose of about 5000 to about 8000 SU/mL (e.g. about 5000 SU, about 5200 SU, about 5400 SU, about 5600 SU, about 5800 SU, about 6000 SU, about 6200 SU, about 6400 SU, about 6600 SU, about 6800 SU, about 7000 SU, about 7200 SU, about 7400 SU, about 7600 SU, about 7800 SU, or about 8000 SU).

In some embodiments, the vials are glass vials. In some embodiments, the vials are fitted with a halobutyl rubber stopper and aluminium seal.

In some embodiments, the kit comprises: (i) a first syringe comprising about 0.5 mL to about 1.0 mL of a composition comprising one or more antigens in a dose of about 300 to about 1500 SU/mL (e.g. about 300 SU, about 400 SU, about 500 SU, about 600 SU, about 700 SU, about 800 SU, about 900 SU, about 1000 SU, about 1100 SU, about 1200 SU, about 1300 SU, about 1400 SU, or about 1500 SU); (ii) a second syringe comprising about 0.5 mL to about 1.0 mL of a composition comprising one or more antigens in a dose of about 2000 to about 4000 SU/mL (e.g. about 2000 SU, about 2100 SU, about 2200 SU, about 2300 SU, about 2400 SU, about 2500 SU, about 2600 SU, about 2700 SU, about 2800 SU, about 2900 SU, about 3000 SU, about 3100 SU, about 3200 SU, about 3300 SU, about 3400 SU, about 3500 SU, about 3600 SU, about 3700 SU, about 3800 SU, about 3900 SU, or about 4000 SU); (iii) a third syringe comprising about 0.5 mL to about 1.0 mL of a composition comprising one or more antigens in a dose of about 5000 to about 8000 SU/mL (e.g. about 5000 SU, about 5200 SU, about 5400 SU, about 5600 SU, about 5800 SU, about 6000 SU, about 6200 SU, about 6400 SU, about 6600 SU, about 6800 SU, about 7000 SU, about 7200 SU, about 7400 SU, about 7600 SU, about 7800 SU, or about 8000 SU); (iv) a fourth syringe comprising about 0.5 mL to about 1.0 mL of a composition comprising one or more antigens in a dose of about 5000 to about 8000 SU/mL (e.g. about 5000 SU, about 5200 SU, about 5400 SU, about 5600 SU, about 5800 SU, about 6000 SU, about 6200 SU, about 6400 SU, about 6600 SU, about 6800 SU, about 7000 SU, about 7200 SU, about 7400 SU, about 7600 SU, about 7800 SU, or about 8000 SU); (v) a fifth syringe comprising about to about 1.0 mL of a composition comprising one or more antigens in a dose of about 5000 to about 8000 SU/mL (e.g. about 5000 SU, about 5200 SU, about 5400 SU, about 5600 SU, about 5800 SU, about 6000 SU, about 6200 SU, about 6400 SU, about 6600 SU, about 6800 SU, about 7000 SU, about 7200 SU, about 7400 SU, about 7600 SU, about 7800 SU, or about 8000 SU); and (vi) a sixth syringe comprising about 0.5 mL to about 1.0 mL of a composition comprising one or more antigens in a dose of about 5000 to about 8000 SU/mL (e.g. about 5000 SU, about 5200 SU, about 5400 SU, about 5600 SU, about 5800 SU, about 6000 SU, about 6200 SU, about 6400 SU, about 6600 SU, about 6800 SU, about 7000 SU, about 7200 SU, about 7400 SU, about 7600 SU, about 7800 SU, or about 8000 SU).

The kit may be for any method of treatment described herein (see section entitled “Methods of treatment”). In some embodiments, the kit comprises instructions for a method of treating allergic rhinitis, allergic conjunctivitis, and/or allergic rhinoconjunctivitis in a subject.

In some embodiments, each composition comprising one or more antigens is a composition according to the present invention (as described in the section entitled “Compositions”).

Compositions

The present invention provides a composition comprising one or more antigens. The composition may be used in the methods or kits of the present invention. The composition may be a vaccine composition or an allergen-specific immunotherapy composition.

The composition may be formulated for subcutaneous injection. In some embodiments, the composition is provided in a vial. In some embodiments, the composition is provided in a syringe. In some embodiments, the composition volume is from about 0.5 mL to about 2.0 mL (e.g. about 0.5 mL, about 0.6 mL, about 0.7 mL, about 0.8 mL, about 0.9 mL, about 1.0 mL, about 1.1 mL, about 1.2 mL, about 1.3 mL, about 1.4 mL, about 1.5 mL, about 1.6 mL, about 1.7 mL, about 1.8 mL, about 1.9 mL, or about 2.0 mL).

Antigens and Allergens

As used herein, an “antigen” may refer to any substance that can be specifically recognized by the adaptive elements of the immune response, i.e. by B cells or T cells, or both. The one or more antigens used in the present invention may be immunogens, i.e. an antigen which activates immune cells to generate an immune response against itself. The one or more antigens may be obtained by recombinant means or peptide synthesis, or from natural sources or extracts and may be derived from any living or non-living organisms. In some embodiments, the one or more antigens are extracted from an antigen-containing material.

In some embodiments, the one or more antigens antigen are allergens. As used herein, an “allergen” may refer to an antigen that provokes an allergy. Common allergens include pollen, dust mites, foods, latex, medications, and insect stings.

The allergen used in the present invention may be derived from any allergy causing substance, such as, but not limited, to pollen (e.g. Bent pollen, Foxtail pollen, Sweet vernal pollen, False oat pollen, Brome pollen, Crested dogstail pollen, Cocksfoot pollen, Fescue pollen, Yorkshire fog pollen, Rye grass pollen, Timothy pollen, Meadow pollen, Cultivated rye pollen, Ragweed pollen, Mugwort pollen, Birch pollen, Alder pollen, Hazel pollen, Olive pollen, Pariateria pollen, Maple (Acer negundo) pollen, Cypress pollen and Japanese Cedar (Cryptomeria japonica) pollen), food, insect venom, mold, animal derived material such as animal fur, and mites such as the house dust mites (e.g., D. farinae or D. pteronyssinus or Blomia tropicalis). In some embodiments, the one or more antigens are derived from an inhalant allergen. As used herein, an “inhalant allergen” may refer to any allergen that may be taken into the body through the respiratory system. Inhalant allergens may include pollen, house dust mites, animal danders and molds.

In some embodiments, the one or more antigens are derived from pollen. In some embodiments, the one or more antigens are derived from grass/rye pollen, weed pollen, and/or tree pollen. In some embodiments, the one or more antigens are derived from grass/rye pollen and/or weed pollen. In some embodiments, the one or more antigens are derived from grass/rye pollen. In some embodiments, the one or more antigens are derived from one or more of: Bent grass pollen, Foxtail pollen, Sweet vernal pollen, False oat pollen, Brome pollen, Dogstail pollen, Cocksfoot pollen, Fescue pollen, Yorkshire Fog pollen, Rye grass pollen, Timothy grass pollen, Meadow grass pollen, and Cultivated Rye pollen. In some embodiments, the one or more antigens are derived from weed pollen. In some embodiments, the one or more antigens are derived from Ragweed pollen. In some embodiments, the one or more antigens are derived from tree pollen. In some embodiments, the one or more antigens are derived from one or more of: Birch pollen, Alder pollen, and Hazel pollen. In some embodiments, the one or more antigens are derived from Birch pollen.

In some embodiments, the composition comprises purified allergen extracts of pollen. In some embodiments, the composition comprises one or more purified allergen extracts of pollen selected from the following grasses: Bent grass, Foxtail, Sweet vernal, False oat, Brome, Dogstail, Cocksfoot, Fescue, Yorkshire Fog, Rye grass, Timothy grass, Meadow grass and Cultivated Rye. In some embodiments, the composition comprises equal amounts of each of the purified allergen extracts. In some embodiments, the composition comprises a purified allergen extracts of Ragweed. In some embodiments, the composition comprises one or more purified allergen extracts of pollen selected from the following trees: Birch, Alder, and Hazel. In some embodiments, the composition comprises a purified allergen extracts of Birch.

In some embodiments, the allergen is extracted into a phenolic buffered saline solution, optionally at about pH 6.5 (optionally containing Sodium Chloride, Potassium Di-Hydrogen

Phosphate, Disodium Phosphate Dodecahydrate, 80% w/w Liquified Phenol and Water for Injections (WFI)) at about 2 to about 8° C., optionally at about 5° C. In some embodiments, the extraction is performed for about 12 to about 30 hours, for about 14 to about 24 hours, or for about 18 hours. In some embodiments, the extract solution is filtered using a 0.2 μm pore size filter.

In some embodiments, the purified allergen extracts are chemically modified and/or adsorbed onto an adsorbant base

Chemical Modifications

In some embodiments, the one or more antigens are chemically modified. In some embodiments, the one or more antigens are chemically modified by reaction with known substances, for example, formaldehyde or glutaraldehyde, which retain or enhance the desired immunogenic properties of the antigen whilst helping to avoid unwanted adverse effects.

Chemical modification of allergen extracts can alter the conformational B cell epitopes and reduces IgE reactivity. In contrast, T-cell epitopes are sequential (i.e. dependent on amino acid sequence rather than protein structure) and the IgG stimulating properties of the original allergens are retained. As it is IgE reactivity that potentially leads to acute allergic reactions, the inclusion of an allergoid, rather than a native extract, can enhance the safety profile of the product, while retaining its potential to induce allergen-specific IgG antibodies.

In some embodiments, the one or more antigens are modified by treatment with glutaraldehyde. Glutaraldehyde is an aldehyde that can cross-link proteins, mainly via protein primary amine moieties from lysine and the N-terminus of the protein. Studies have shown that such allergoids had greatly reduced IgE immunoreactivity, whilst retaining the potential to induce allergen-specific IgG antibodies as compared to unmodified allergens.

The modification process may involve mixing of aqueous allergen extracts with glutaraldehyde. In some embodiments, the final composition is substantially free of unbound glutaraldehyde as confirmed post co-precipitation washing. In some embodiments, quenching of the glutaraldehyde with glycine is performed.

Adsorbant Bases

In some embodiments, the composition comprises an adsorbant base. The one or more antigens may be adsorbed onto the adsorbant base. As used herein, an “adsorbant base” may act as a short-term depot thereby delaying the bio-availability of allergenic proteins and enhancing IgG antibody induction.

In some embodiments, the adsorbant base is an amino acid. In some embodiments, the amino acid used in the invention has a water solubility of about 1.1 or less g/100 ml H₂O at In some embodiments, the amino acids is tyrosine or tryptophan. Pharmaceutically acceptable derivatives of these amino acids are also included within the scope of the present invention, such as benzyl-O-octadecanoyl-L-tyrosine.

In some embodiments, the adsorbant base is tyrosine. Tyrosine (symbol Tyr or Y) or 4-hydroxyphenylalanine is an amino acid with a polar side group. Suitably, the tyrosine is L-tyrosine. The structure of L-tyrosine is shown below:

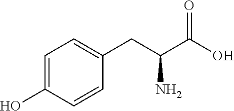 [SMILES: N[C@@H](Cc1ccc(O)cc1)C(=O)O]

An advantage of using L-tyrosine as the adsorbant base is that it is readily metabolized as opposed to other adsorbants, and therefore less likely to form persistent lesions at the site of injection.

In some embodiments, the L-tyrosine is in the form of microcrystalline tyrosine (MCT). Microcrystalline tyrosine is a crystallized tyrosine that contains microscopic crystals or particles.

The crystals or particles may have size of about 0.1 microns to about 100 microns or about 1 microns to about 100 microns. Particle size may be determined by any suitable method known to those of skill in the art. Particle size may be determined by optical microscopy, for example light microscopy or fluorescence microscopy. Particle size may be determined by electron microscopy, for example transmission electron microscopy.

The microcrystalline tyrosine may have a median particle length of about 10 to about 100 microns (e.g. about 10 microns, about 20 microns, about 30 microns, about 40 microns, about 50 microns, about 60 microns, about 70 microns, about 80 microns, about 90 microns, or about 100 microns). In some embodiments, the microcrystalline tyrosine has a median particle length of about 10 to about 50 microns, about 30 to about 50 microns, or about 40 microns. The microcrystalline tyrosine may have a median particle width of about 1 to about 10 microns (e.g. about 1 micron, about 2 microns, about 3 microns, about 4 microns, about 5 microns, about 6 microns, about 7 microns, about 8 microns, about 9 microns, or about 10 microns). In some embodiments, the microcrystalline tyrosine has a median particle width of about 2 to about 6 microns, about 4 to about 6 microns, or about 5 microns. In some embodiments, the microcrystalline tyrosine has a median particle length of about 10 to about 100 microns and a median particle width of about 1 to about 10 microns.

The microcrystalline tyrosine may have substantially the same Fourier-transform infrared spectroscopy (FTIR) spectra as an L-tyrosine standard. The microcrystalline tyrosine may have a FTIR band at about 2077 cm⁻¹. The microcrystalline tyrosine may have the substantially the same x-ray powder diffraction (XRD) pattern as an L-tyrosine standard. The microcrystalline tyrosine may have the substantially the same thermogravimetric analysis (TGA) curve as an L-tyrosine standard. As used herein, an “L-tyrosine standard” may be a certified reference material produced and certified in accordance with ISO/IEC 17025 and ISO 17034.

The composition may comprise the adsorbant base (e.g. L-tyrosine) in any suitable amount. In some embodiments, the composition comprises the adsorbant base (e.g. L-tyrosine) in an amount of about 0.5% w/v to about 5% w/v (e.g. about 0.5% w/v, about 1.0% w/v, about 1.5% w/v, about 2.0% w/v, about 2.5% w/v, about 3.0% w/v, about 3.5% w/v, about 4.0% w/v, about 4.5% w/v, or about 5.0% w/v). In some embodiments, the composition comprises the adsorbant base (e.g. L-tyrosine) in an amount of about 2% w/v. In some embodiments, the composition comprises the adsorbant base (e.g. L-tyrosine) in an amount of about 3% w/v.

Adjuvants

In some embodiments, the composition comprises one or more adjuvants. As used herein, an “adjuvant” may enhance the effect of the allergen-specific immunotherapy.

In some embodiments, the adjuvant is a TH1-inducing adjuvant. As used herein, a “TH1-inducing adjuvant” may refer to an adjuvant that enhances the TH1 response to an antigen. The effectiveness of an adjuvant as a TH1-inducing adjuvant may be determined by determining the profile of antibodies directed against an antigen resulting from administration of this antigen in vaccines which are also comprised of the various adjuvants. The presence of the adjuvant may alter the balance of the immune response in allergic patients away from an IgE-related Th2 response.

In some embodiments, the adjuvant is a modified lipopolysaccharide. Enterobacterial lipopolysaccharides (LPS) is a powerful immunostimulant, however, it can also illicit harmful and sometimes fatal responses. It is now known that the endotoxic activities associated with LPS result from its lipid A component. Accordingly, the present invention preferably uses a detoxified derivative of lipid A. Ribi ImmunoChem produced a derivative of lipid A originally known as refined detoxified endotoxin (RDE) but which has become known as monophosphoryl lipid A (MPL). As described in U.S. Pat. No. 4,912,094, MPL is produced by refluxing LPS or lipid A obtained from heptoseless mutants of gram negative bacteria (e.g. Salmonella sp.) in mineral acid solutions of moderate strength (e.g. 0.1N HCl) for a period of around 30 minutes. This treatment results in loss of the phosphate moiety at position 1 of the reducing-end glucosamine. In addition the core carbohydrate is removed from the 6′ position of the non-reducing glucosamine during this treatment.

In some embodiments, a modified LPS or lipid A is used in which the detoxified lipid A retains the core moiety attached to the 6′ position of non-reducing glucosamine. Such derivatives of LPS and lipid A are also described in U.S. Pat. No. 4,912,094. In more detail, U.S. Pat. No. 4,912,094 discloses a modified lipopolysaccharide which is obtained by the method of selectively removing only the 3-hydroxymyristic acyl residue of lipopolysaccharide that is ester-linked to the reducing-end glucosamine at position 3′ of said lipopolysaccharide, which comprises subjecting said lipopolysaccharide to alkaline hydrolysis. Such de-O-acylated monophosphoryl lipid A (MPL), diphosphoryl lipid A (DPL) and LPS may be used in the present invention. Thus, in some embodiments, the adjuvant is MPL, DPL or LPS in which the position 3′ of the reducing end glucosamine is de-O-acylated. These compounds may be known as 3-DMPL, 3-DDPL and 3-DLPS, respectively.

U.S. Pat. No. 4,987,237 describes derivatives of MPL having the formula:

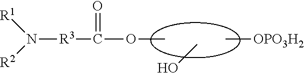

wherein R¹ and R² are H, R³ is straight or branched chain hydrocarbon composed of C, H and optionally O, N and S, which if more than one atom may be the same or different, wherein the total number of C atoms does not exceed 60, and the circle represents an MPL nucleus.

Alternatively the MPL Derivative May have the Formula

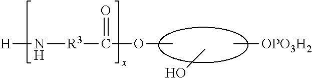

wherein the segment of the derivative represented by

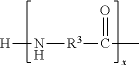

contains 2-60 C atoms and wherein R³ is straight or branched chain hydrocarbon composed of C, H and optionally O, N and S, which if more than one atom may be the same or different, and x is a minimum of 1 and can be any whole number such that the total number of C atoms in all x segments does not exceed 60, and wherein the chemical structure of each R³ may be the same or different in each such segment and wherein the circle represents an MPL nucleus.

All such derivatives or salts of LPS or lipid A which are or become available may be used in the present invention. Suitable derivatives and salts include ones which are pharmaceutically acceptable.

In some embodiments, the adjuvant is 3-O-desacyl-4′-monophosphoryl lipid A (see e.g. Cluff, C. W., 2009. In Lipid A in cancer therapy (pp. 111-123). Springer, New York, NY) which may also be known as 3-deacylated monophosphoryl lipid A.

In some embodiments, the derivatives or salts of LPS or lipid A (e.g. MPL, 3-DMPL or a derivative or salt thereof) has a mean particle size of about 500 nm or less, about 400 nm or less, about 300 nm or less, about 200 nm or less, about 150 nm or less, or about 144 nm or less. In some embodiments, the derivatives or salts of LPS or lipid A (e.g. MPL, 3-DMPL or a derivative or salt thereof) has a mean particle size of about 90 nm to about 120 nm. Particle size may be determined by any suitable method known to those of skill in the art.

The composition may comprise the adjuvant (e.g. MPL, 3-DMPL or a derivative or salt thereof) in any suitable amount. In some embodiments, the composition comprises the adjuvant (e.g. MPL, 3-DMPL or a derivative or salt thereof) in an amount of about 25 μg/mL to about 100 μg/mL (e.g. about 25 μg/mL, about 30 μg/mL, 25 μg/mL, 35 μg/mL, 40 μg/mL, 45 μg/mL, 50 μg/mL, 60 μg/mL, 70 μg/mL, 80 μg/mL, 90 μg/mL, or 100 μg/mL). In some embodiments, the composition comprises the adjuvant (e.g. MPL, 3-DMPL or a derivative or salt thereof) in an amount of about 50 μg/mL.

Other Components

The composition may comprise any other suitable components such as a surfactant, a preservative, a buffering agent, a tonicity agent and a diluent.

Suitable components may include any pharmaceutically acceptable component. By “pharmaceutically acceptable” is included that the formulation is sterile and pyrogen free. The components should be “acceptable” in the sense of being compatible with the one or more antigens and not deleterious to the recipients thereof.

Surfactant

In some embodiments, the composition comprises one or more surfactants. As used herein, a “surfactant” may refer to a substance that lowers the interfacial tension between two liquids, between a gas and a liquid, or between a liquid and a solid. Surfactants may act as detergents, wetting agents, emulsifiers, foaming agents, or dispersants.

Any suitable surfactant may be used. In some embodiments, the composition comprises dipalmitoyl phosphatidylcholine. Dipalmitoyl phosphatidylcholine (DPPC) is a phospholipid consisting of two C16 palmitic acid groups attached to a phosphatidylcholine head-group.

In some embodiments, the surfactant (e.g. DPPC) is present in an amount of about 0.001 to about 0.01 mg/ml (e.g. about 0.001 mg/ml, about 0.002 mg/ml, about 0.003 mg/ml, about 0.004 mg/ml, about 0.005 mg/ml, about 0.006 mg/ml, about 0.007 mg/ml, about 0.008 mg/ml, about 0.009 mg/ml or about 0.001 mg/ml). In some embodiments, the surfactant (e.g. DPPC) is present in an amount of about 0.006 mg/ml.

Preservative

In some embodiments, the composition comprises one or more preservative. As used herein, a “preservative” may refer to a substance that is used to preserve compositions against decay.

Any suitable preservative may be used. In some embodiments, the composition comprises phenol. Liquefied phenol may be used to keep bacteria from growing in and contaminating the vaccine solutions.

In some embodiments, the preservative (e.g. phenol) is present in an amount of about 1 to about 10 mg/ml (e.g. about 1 mg/ml, about 2 mg/ml, about 3 mg/ml, about 4 mg/ml, about 5 mg/mi, about 6 mg/ml, about 7 mg/mi, about 8 mg/mi, about 9 mg/mi or about 10 mg/mi). In some embodiments, the preservative (e.g. phenol) is present in an amount of about 5 mg/ml.

Tonicity Agent and Buffers

In some embodiments, the composition comprises one or more tonicity agent and/or one or more buffering agent.

As used herein, a “tonicity agent” may be used to make a solution isotonic with blood. As used herein, a “buffering agent” may be used to regulate the pH of a solution. In some embodiments, the composition has a pH of about 5 to about 9, a pH of about 6 to about 8, or a pH of about 7.

Any suitable tonicity agent may be used. In some embodiments, the composition comprises sodium chloride. In some embodiments, the composition comprises sodium chloride in an amount of from about 5 to about 20 mg/mL (e.g. about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, or about 20 mg/mL). In some embodiments, the composition comprises sodium chloride in an amount of about 9.0 mg/mL.

Any suitable buffering agent may be used. In some embodiments, the composition comprises disodium phosphate dodecahydrate and sodium dihydrogen phosphate dihydrate. In some embodiments, the composition comprises disodium phosphate dodecahydrate and sodium dihydrogen phosphate dihydrate in an amount of about 1 to about 10 mg/mL (e.g. about 1 mg/ml, about 2 mg/ml, about 3 mg/ml, about 4 mg/ml, about 5 mg/ml, about 6 mg/ml, about 7 mg/ml, about 8 mg/ml, about 9 mg/ml or about 10 mg/ml). In some embodiments, the composition comprises disodium phosphate dodecahydrate and sodium dihydrogen phosphate dihydrate in an amount of about 4.0 mg/mL.

Diluent

In some embodiments, the composition comprises one or more diluent. Any suitable diluent may be used. In some embodiments, the diluent is water. The diluent may be present in any quantity to give the desired final volume.

Method of Manufacture

Any suitable manufacturing process may be used to prepare the composition of the present invention. In some embodiments, the manufacturing process is Good manufacturing Practice (GMP)-compliant. The manufacturing processes may be designed to remove IgE reactivity, whilst retaining IgG potency.

In some embodiments, a composition comprising one or more antigens adsorbed to an amino acid is prepared by a method comprising:
(i) mixing a solution of one or more antigens with a solution of the amino acid in an aqueous acid whilst neutralising the mixture of solutions, thereby forming an adsorbate comprising the one or more antigens and the amino acid;
(ii) separating the adsorbate into a buffer by cross-flow filtration thereby forming said composition; and
(iii) recovering said composition.

In some embodiments, steps (i) to (iii) are performed in a sterile environment and within a closed system.

In some embodiments, a composition comprising one or more antigens adsorbed to an amino acid is prepared by a method comprising:
(i) modifying the one or more antigens with glutaraldehyde;
(ii) removing excess glutaraldehyde using cross-flow filtration to form a modified solution;
(iii) mixing the modified solution with a solution of the tyrosine in an aqueous acid whilst neutralising the mixture of solutions, thereby forming an adsorbate comprising the modified allergens and the tyrosine;
(iv) separating the adsorbate into a buffer by cross-flow filtration thereby forming said composition; and
(v) recovering said composition;

In some embodiments, steps (iii) to (v) are performed in a sterile environment and within a closed system

In some embodiments, a composition comprising one or more antigens adsorbed to an amino acid is prepared by a method comprising:
(i) extracting the one or more antigens into solution to form an extract solution;
(ii) filtering the extract solution to remove solids;
(iii) performing cross-flow filtration and isolating the retentate comprising the antigen;
(iv) modifying the one or more antigens with glutaraldehyde;
(v) removing excess glutaraldehyde using cross-flow filtration to form a modified solution;
(vi) sterile filtering the modified solution;
(vii) mixing the modified solution with a solution of tyrosine in an aqueous acid whilst neutralising the mixture of solutions, thereby forming an adsorbate comprising the modified allergen and the tyrosine;
(viii) separating the adsorbate into a buffer by cross-flow filtration thereby forming said composition; and
(ix) recovering said composition

In some embodiments, steps (vii) to (ix) are performed in a sterile environment and within a closed system. In some embodiments, step (i) is performed using a phenolic buffered solution at about 2 to about 8° C. for about 18 hours.

In some embodiments, removing the excess glutaraldehyde using cross-flow filtration is performed using a membrane with a 5 to 10 kDa (e.g. about 5 kDa, about 6 kDa, about 7 kDa, about 8 kDa, about 9 kDa, or about 10 kDa) molecular weight cut-off. In some embodiments, separating the adsorbate comprising the antigen and the amino acid using cross-flow filtration is performed using a poly-sintered stainless steel filter. In some embodiments, the poly-sintered stainless steel filter is a 5 μm pore size filter.

In some embodiments, the adsorbate comprising the antigen and the amino acid is formed by mixing the antigen with amino acid in HCl having a molarity of about 3.8M whilst neutralising the mixture with NaOH having a molarity of about 3.2M.

In some embodiments, said composition is diluted to the desired concentration for subcutaneous use. In some embodiments, an adjuvant is added to said composition.

Preparation

In some embodiments, the composition of the present invention is prepared by mixing an aqueous solution of the antigen with a solution of the amino acid in a strong aqueous acid (e.g. hydrochloric acid) and neutralizing the mixture (e.g. with sodium hydroxide), thereby co-precipitating the amino acid and antigen.

In some embodiments, an aqueous solution of the antigen (optionally at about pH 6.3 to 7.2) is mixed with a solution of the amino acid in a strong aqueous acid. In some embodiments, the strong acid is an inorganic acid, for example hydrochloric acid. In some embodiments, the strong acid is hydrochloric acid having a molarity of about 3.5M to about 4.5M, or about 3.8M. In some embodiments, the solution of antigen used in this step contains up to about antigen protein. In some embodiments, the solution of amino acid used is about 24% w/v.

In some embodiments, the resulting mixture of solutions of antigen and amino acid is neutralized (e.g. with sodium hydroxide). In some embodiments, the sodium hydroxide has a molarity of about 3M to about 3.5M, preferably about 3.2M. It is desirable that, at no time, or at least no prolonged time, during the neutralization does the pH of the solution deviate from equilibrium. This condition can be met by vigorous stirring of the solution and by the use only of the required amount of base, if desired. Various buffering agents such as buffered saline solution can usefully be added to the solutions of antigen to assist in pH control during mixing and neutralizing stages.

A particularly useful method of carrying out the neutralization is for separate streams of the solution of amino acid and neutralizing base to be run into the solution of antigen. The rates of flow of the added solutions are controlled by equipment which regulates the flow of one or both of the solutions so that the pH of the reaction mixture remains substantially constant at a predetermined level. Optimum results are usually obtained by pH control within the range of about 6.5 to about 7.5 or about 6.8 to about 7.2, though the precise pH may vary according to the nature of the antigen. In some embodiments, at no time, or at least no prolonged time, during the neutralization does the pH move from equilibrium i.e., move outside the pH range of about 6.5 to about 7.5 or about 6.8 to about 7.2. The result of the neutralization is the immediate precipitation of the amino acid, within and/or upon which the solution of antigen is occluded and/or adsorbed.

Cross-Flow Filtration

A method that has been useful in the fractionation of various particles is cross-flow filtration or tangential-flow filtration (TFF). Cross-flow filtration is a separation process that uses membranes to separate components in a liquid solution or suspension on the basis of size or molecule weight differences. In cross-flow filtration, the solution or suspension to be filtered is passed across the surface of the membrane in a cross-flow mode. The driving force for filtration is the transmembrane pressure. The velocity at which the filtrate is passed across the membrane surface also controls the filtration rate and helps prevent clogging of the membrane. Because cross-flow filtration recirculates retentate across the membrane surface, membrane fouling is minimized, a high filtration rate is maintained, and product recovery is enhanced.

Cross-flow filtration devices generally comprise a pump, a feed solution reservoir, a filtration module and conduits for connecting these elements. In use, the feed solution is directed from the feed solution reservoir to the filtration module while the retentate from the filtration module is recirculated from the filtration module to the feed solution reservoir until the desired volume of retentate is obtained.

In some embodiments, a cross-flow filtration is used to separate the adsorbate comprising the modified antigen and the amino acid using about a 5 μm pore size poly-sintered stainless steel filter maintaining pressure between about 1.1 to about 1.3 bar.

Closed System

A closed system is an isolated system that prevents exposure of the composition to the environment outside of the system. The composition is only exposed to the immediate environment of tubing and machine components that make up the closed system. A closed system may prevent contamination of the composition. It achieves this by ensuring that the composition is sealed off from the environment external to the system, preventing contaminants from entering the system. Pipework for all material transfer may be Clean in Place (CIP)/Steam in Place (SIP).

### EXAMPLES

The invention will now be further described by way of Examples, which are meant to serve to assist one of ordinary skill in the art in carrying out the invention and are not intended in any way to limit the scope of the invention.

### Example 1— Efficacy of an Extended Posology in the Context of Subcutaneous Allergen-Specific Immunotherapy

Exploratory Field Study Design

An exploratory field study was carried out to explore the efficacy and safety of a subcutaneous allergen-specific immunotherapy, Pollinex Quattro (PQ) Grass 27600 SU, in subjects with seasonal allergic rhinitis and/or rhinoconjunctivitis induced by grass pollen exposure.

PQ Grass contains selectively purified allergen extracts of pollen (grass/rye, tree or weed). The allergens have been modified into allergoids by treatment with glutaraldehyde and are adsorbed onto L-tyrosine. The allergen extracts are standardised (in SU, Standardised Units) by biochemical methods and characterised, to provide a constant quality of allergen content and allergen activity. Each 1.0 mL dose contains 50 μg of the adjuvant Monophosphoryl Lipid A (MPL).

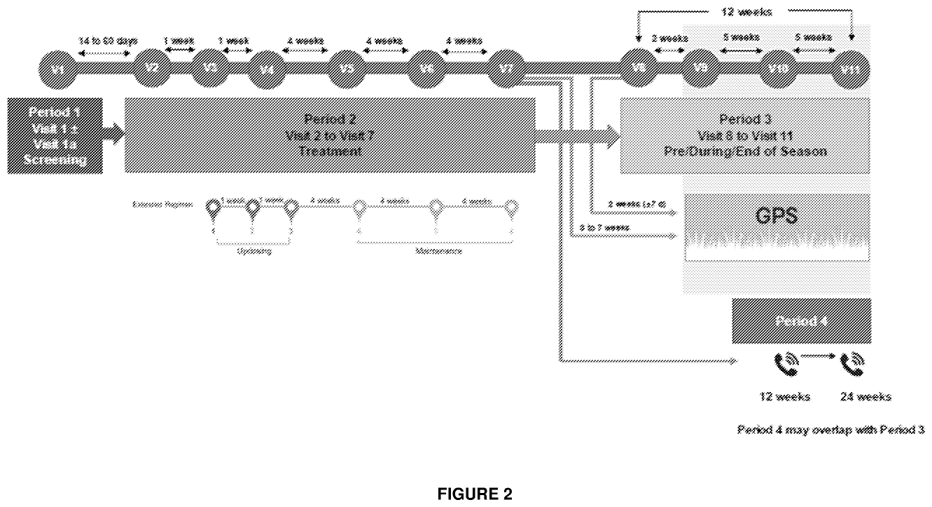

119 subject were enrolled, with a mean age of 33.15 and randomised to one of four treatment arms (dosing schedules are summarised in Table 1):
PQ Grass conventional posology (N=41): 6 once weekly injections of PQ grass (900 SU, 2700 SU, 4×6000 SU) (see FIG. 2A)
PQ Grass extended posology (N=40): 3 once weekly injections of PQ grass (900 SU, 2700 SU, 6000 SU) and 3 once monthly injections (3×6000 SU) (see FIG. 2B)
Placebo containing MCT (N=20): microcrystalline tyrosine (MCT) is a depot adjuvant which extends the bioavailability of vaccines and enhances IgG induction
Placebo without MCT (N=18): normal saline

TABLE 1
summary of dosage schedules (doses in SU/ml)

| | 1st | 2nd | 3rd | 4th | 5th | 6th | 7th | 8th | 9th | 10th | Total |
|---|---|---|---|---|---|---|---|---|---|---|---|
| | inj | inj | inj | inj | inj | inj | inj | inj | inj | inj | dose |
| PQ Grass | 0* | 0* | 0* | 0* | 900 | 2700 | 6000 | 6000 | 6000 | 6000 | 27600 |
| (conventional posology) | | | | | | | | | | | |
| PQ Grass (extended | 900 | 2700 | 6000 | 6000 | 0* | 6000 | 0* | 0* | 0* | 6000 | 27600 |
| posology) | | | | | | | | | | | |
| Placebo containing MCT | 0\hat{ } | 0\hat{ } | 0\hat{ } | 0\hat{ } | 0 | 0 | 0 | 0 | 0 | 0 | 0 |
| Placebo without MCT | 0 | 0 | 0 | 0 | 0 | 0 | 0 | 0 | 0 | 0 | 0 |

The double-blind, randomised, placebo-controlled, parallel group design is the gold standard for clinical studies. Randomisation and double-blinding were employed to minimise bias. Placebo was selected as the appropriate comparator for the Phase II/III assessment of efficacy and safety in order to optimise the sensitivity, reduce bias, and maintain the scientific rigor of the study. Two control products were used in the study: placebo containing MCT (indistinguishable from the active treatment), containing 2% (w/v) L-tyrosine and 0.5% (w/v) phenol and placebo without MCT (distinguishable from the active treatment and the placebo containing MCT), containing buffered saline solution and 0.5% (w/v) phenol. Each 1.0 mL placebo injection was administered subcutaneously.

The exploratory field study design is summarised in FIG. 2. The study included 4 periods, encompassing 15 visits to the study site, an additional visit in the event of a prolonged screening period, unscheduled visit for confirmatory conjunctival provocation test (CPT) (if required), electronic diary (eDiary) retraining, relief medication resupply, adverse event (AE) follow-up, etc. as needed. Depending on any Coronavirus Disease 2019 (COVID-19) related restrictions, Visit 13 and Visit 14 could be performed remotely with study sites providing subjects with enough relief medication to cover them through this period. Subjects not meeting all eligibility criteria at Visit 1 or Visit 1a could be re-screened once.

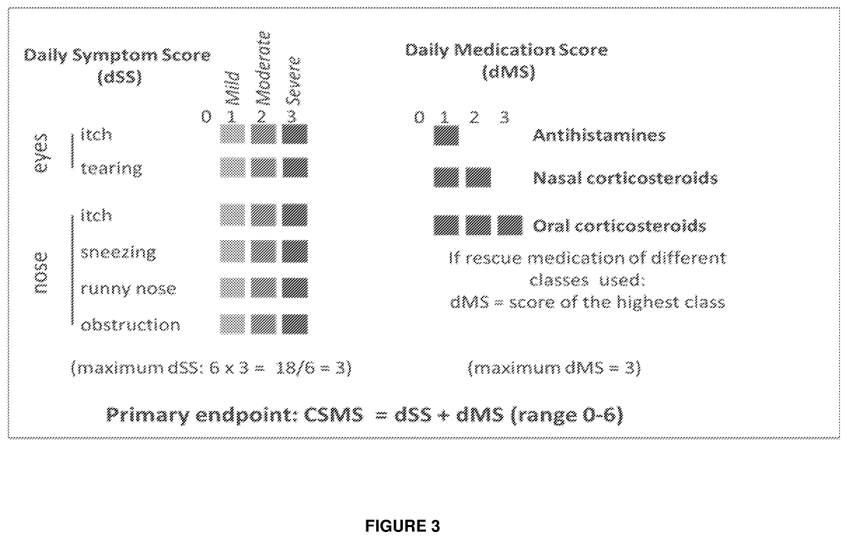

The primary outcome measure was the combined symptom medication score (CSMS) which was averaged over the peak grass pollen season (GPS) and calculated from the daily symptom score (dSS) and daily medication score (dMS) (see FIG. 3).

The symptom assessment was based on the recommendations of the EAACI (Pfaar, O., et al., 2014. Allergy, 69(7), pp. 854-867). Symptom severity was self-recorded by the subject in an eDiary on a daily basis during Period 3. This was a “reflective” assessment that evaluated the symptoms over the previous 24 hours. 6 individual symptoms were assessed as follows: conjunctival symptoms (2 items): itchy/red eyes (collected as 2 separate symptoms with the maximum used for analysis), watery eyes; and nasal symptoms (4 items): blocked nose, runny nose, itchy nose, sneezing. Each of the 6 symptoms was scored on a 4-point severity scale (0=no symptoms, 1=mild symptoms, 2=moderate symptoms, 3=severe symptoms). The dSS for CSMS was calculated as the sum of the scores for the 6 individual symptoms, divided by 6 (i.e., ranging from 0 to 3).

The medication scoring was based on the recommendations of EAACI. At Visit 12, the subject was provided with open-label relief medication to be used as needed for treatment of their allergic rhinitis and/or rhinoconjunctivitis. The subject was instructed to strictly follow the stepwise usage of relief medication, (re-) starting with Step 1 relief medication on each applicable day, and to report medication taken via the eDiary. The medication was rated on a 4-point scale: 0=no relief medication used; 1=oral antihistamine/ocular antihistamine (Step 1 medication); 2=intranasal corticosteroids (Step 2 medication) with Step 1 medication(s); 3=oral corticosteroids (Step 3 medication) with Step 1 and Step 2 medications. For scoring the CSMS a subject was assigned 1 score (i.e., 1, 2 or 3) per day based on at least 1 dose of the medication of the highest step taken that day, regardless if more than 1 medication from different steps had been taken. For example, a subject taking antihistamine eye drops (Step 1), an antihistamine tablet (Step 1) and intranasal corticosteroids (Step 2) would have been allotted a score of 2 for the Step 2 medication taken.

Secondary outcome measures included: CSMS averaged over the entire (or truncated) GPS; Total combined score (TCS) averaged over the peak GPS; TCS averaged over entire (or truncated) GPS; dSS averaged over the peak and entire (or truncated) GPS; dMS averaged over the peak and entire (or truncated) GPS; the probability of well days and severe days during the peak and entire (or truncated) GPS; Rhinoconjunctivitis quality of life questionnaires (RQLQs) measured within the GPS; and serum IgG4 responses.

The daily TCS was the sum of the dSS and dMS calculated from the data recorded in the eDiary. The dSS for total combined score (TCS) was calculated as the sum of the scores for the 6 individual symptoms (i.e., ranging from 0 to 18). The medication was rated on a 4-point scale: No relief medication used=0; Oral antihistamine=each tablet corresponded to a score of 6 with a maximum daily score of 6; Ocular antihistamine=each drop corresponded to a score of 1.5 per eye with a maximum daily score of 6; Intranasal corticosteroid=each spray corresponded to a score of 2 with a maximum daily score of 8. For scoring of the TCS dMS, subjects who took oral corticosteroids were to be allocated the maximum daily score of

Rhinoconjunctivitis quality of life was assessed using the Rhinoconjunctivitis Quality of Life Questionnaire with standardised activities (RQLQ[S]). Subjects were issued the questionnaire at Visits 2 (baseline RQLQ), 13 and 14.

A “well day” was defined based on CSMS as a day with: No use of relief medication on the particular day, and, a total symptom score out of 18 (which corresponds to a dSS of <0.34 for CSMS). A “severe day” was based on CSMS and defined as a day with a symptom score of 3 in any of the 6 rhinitis/rhinoconjunctivitis symptoms. Well days and severe days were assessed during the peak GPS and total GPS.

Grass-specific IgG4 were assessed at screening, Visit 12 and Visit 15. Antibody assay was performed by a central laboratory.

In addition to the primary and secondary outcome measures, immunological and clinical biomarker data was collected and analysed, including grass-specific nasal IgA2, IgG and

IgG4, allergen-driven Th2 and Th2A proliferation, and T follicular helper (Tfh) cells proliferation and induction. Nasal samples for grass-specific IgA2, IgG and IgG4 were collected at Visit 1, Visit 12 and Visit 15. Blood samples were taken at selected clinical sites at Visit 1, Visit 12 and Visit 15.

Exploratory Field Study Results

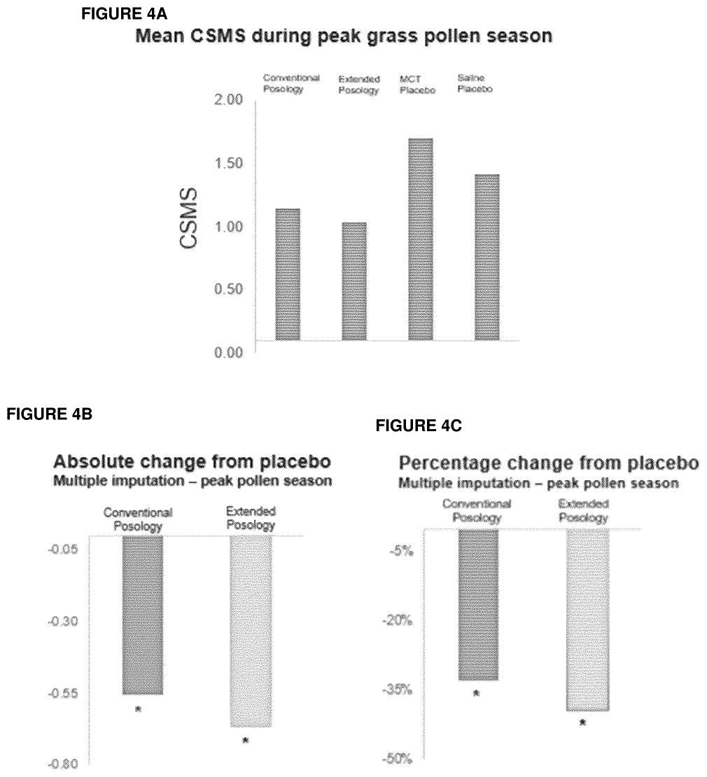

Both the extended posology and the conventional posology demonstrated clinical relevant and statistically significant results compared to the MCT placebo group (see Table 2 and FIGS. 4A-4C). Both exceeded standard criteria for subcutaneous immunotherapy. For example, the Paul-Ehrlich-Institute (PEI) criterion is that a minimal clinically important difference (MCID) is a 30% reduction in CSMS. The result was consistent for all sensitivity analyses. The absolute reduction in CSMS compared to placebo was 0.67 points (80% CI: and 0.56 points (80% CI: 0.23-0.90) for the extended and conventional regimens, respectively.

TABLE 2
mean CSMS during peak grass pollen season

| | | Mean | | |
|---|---|---|---|---|
| | | Difference | | |
| | | [percentage | | |
| | | (absolute)] | | |
| | | versus | 80% Confidence | |
| | N | MCT Placebo | Intervals | P-value |
| PQ Grass | 41 | −33.1% (−0.56) | −48.8% to −17.4% | 0.0325 |
| (conventional) | | | | |
| PQ Grass | 40 | −39.5% (−0.67) | −54.7% to −24.4% | 0.0112 |
| (extended) | | | | |

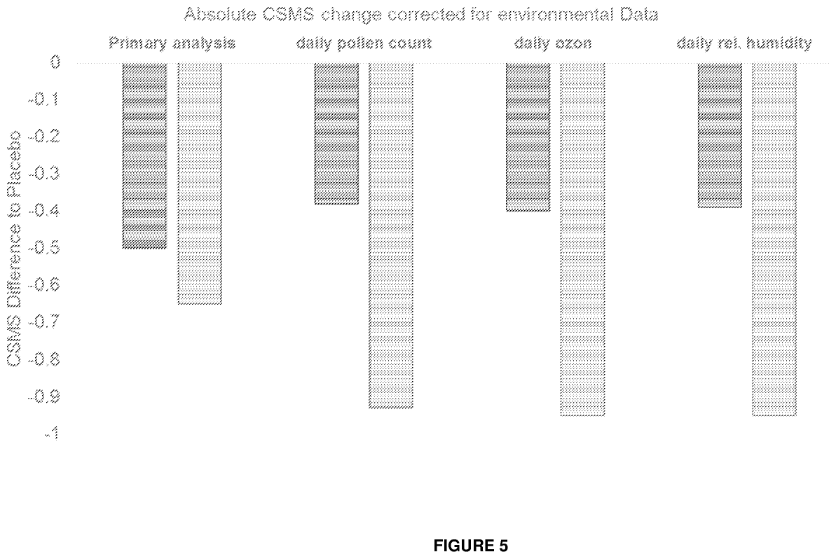

The extended posology demonstrated a greater mean difference compared to the conventional posology. This difference was more pronounced when correcting for environmental effects (see FIG. 5).

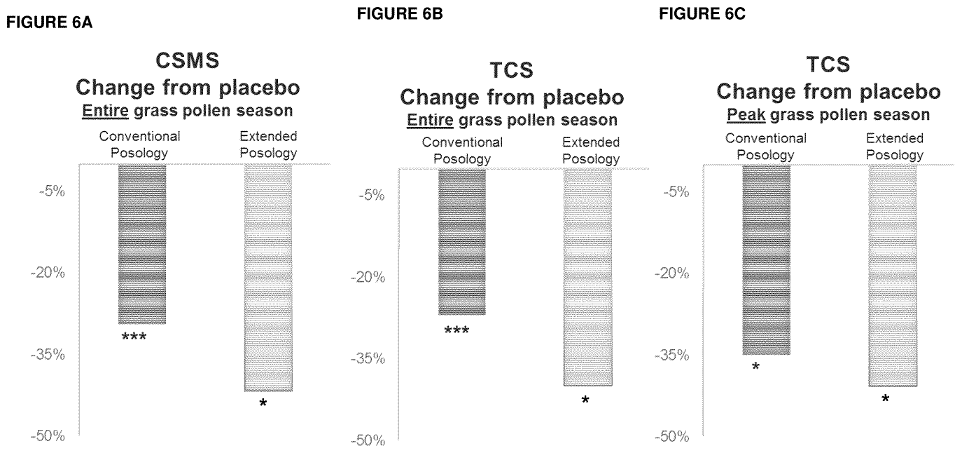

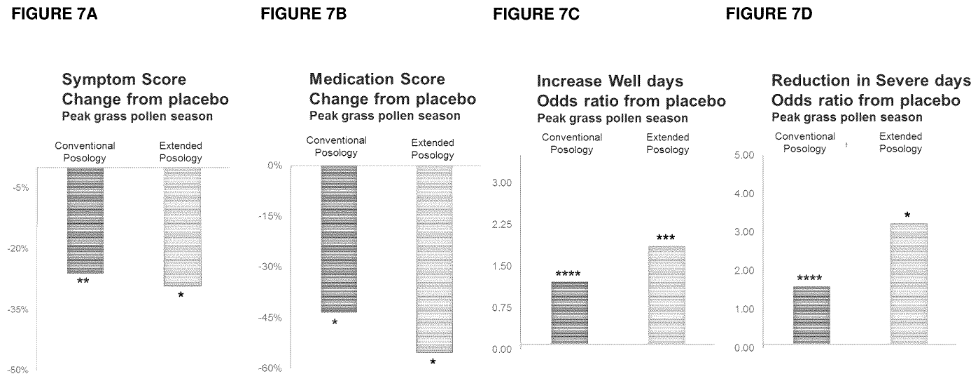

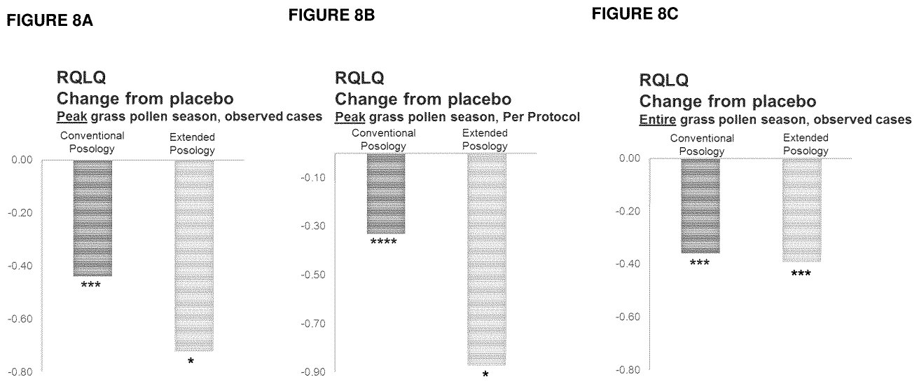

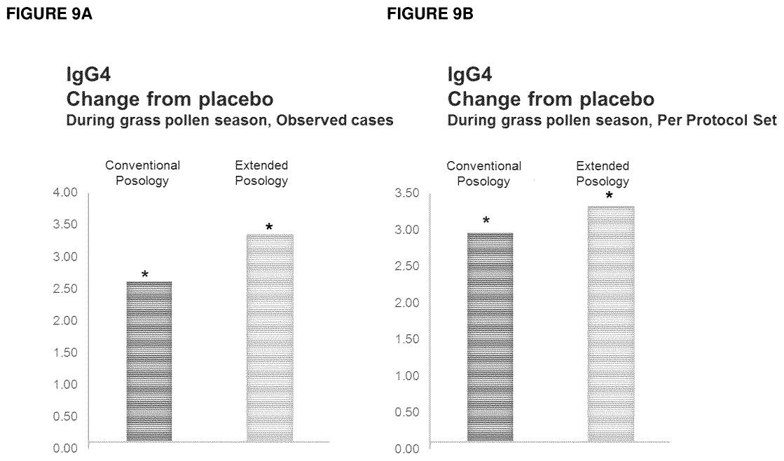

The greater treatment effect of the extended posology compared to the conventional posology was also observed for the main secondary outcomes:
The results for CSMS over the entire grass pollen season; TCS over the entire grass pollen season; and TCS over the peak grass pollen season are shown in FIGS. 6A-6C. Compared to MCT placebo, CSMS averaged over the entire GPS [−25.1% for the conventional posology and −38.8% for the extended posology], TCS averaged over the peak GPS [−35.0% for the conventional posology and −40.8% for the extended posology] and TCS averaged over the entire GPS [−26.9% for the conventional posology and −39.9% for the extended posology] each showed more pronounced treatment effect for the extended posology.
The results for dSS over the peak grass pollen season; dMS over the peal grass pollen season; and the probability of well days and severe days during the peak grass pollen season are shown in FIGS. 7A-7D. The respective odds ratio (OR) compared to placebo was 2.45 for the extended regimen and 1.86 for the conventional regimen. The mean number of well days during the peak GPS was higher for the extended regimen (6.46 days) compared to the conventional regimen (4.73 days). Similarly, the mean number of severe days was lower for the extended regimen (1.03 days) compared to the conventional regimen (2.07 days). The respective OR compared to placebo was 0.31 for the extended regimen and 0.63 for the conventional regimen.
The results for RQLQ over peak and entire grass pollen season are shown in FIGS. 8A-8C. There was more pronounced improvement in RQLQ averaged over the peak GPS for the extended posology (−0.72 points, −37.9%) compared to the conventional posology (−0.44 points, −22.9%) against the placebo containing MCT group. There was a strong positive correlation between the CSMS and the RQLQ-S measured during the entire GPS. A 0.5-point improvement in RQLQ-S was calculated to correspond to an absolute CSMS improvement of 0.34 points.
The results for change in IgG4 are shown in FIGS. 9A-9B. Statistically significant changes were observed in allergen specific IgG4 (from baseline to preseason measurement) for both the conventional and extended posologies and this was more pronounced for the extended posology compared to placebo containing MCT (LS mean±SE: +2.61±0.944, p=0.0068 and LS mean±SE: +3.34±0.946, p=0.0006, respectively). At the end of grass pollen season grass-specific serum IgG4 remained elevated compared to placebo.

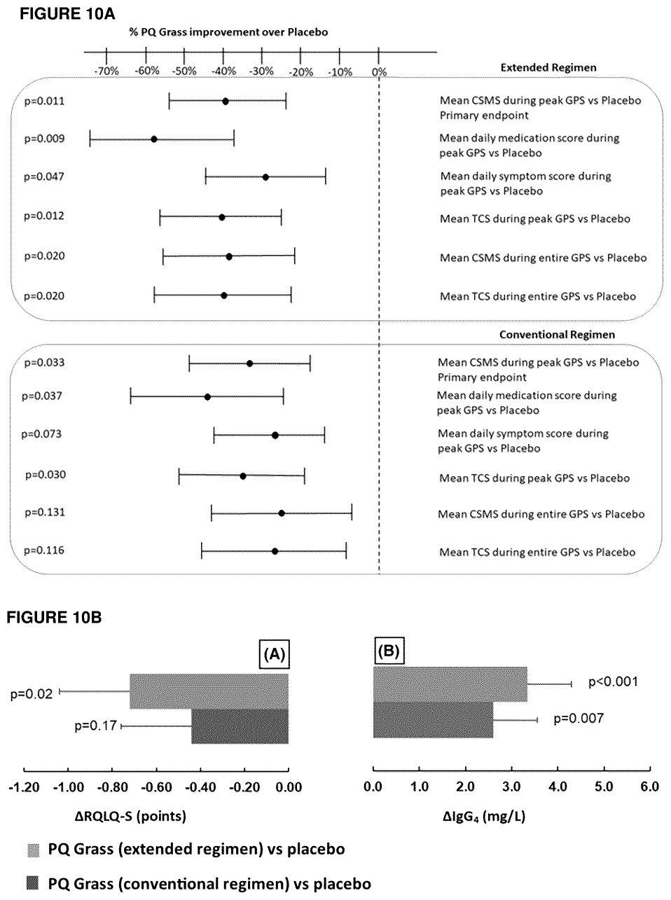

The greater treatment effect of the extended posology compared to the conventional posology is summarised in FIG. 10A-B. There were no statistically significant differences observed in any of the primary and/or secondary efficacy endpoints between both placebo groups.

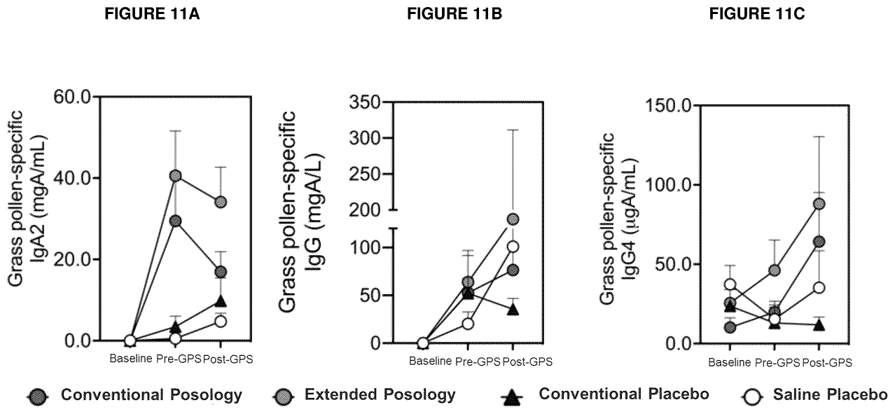

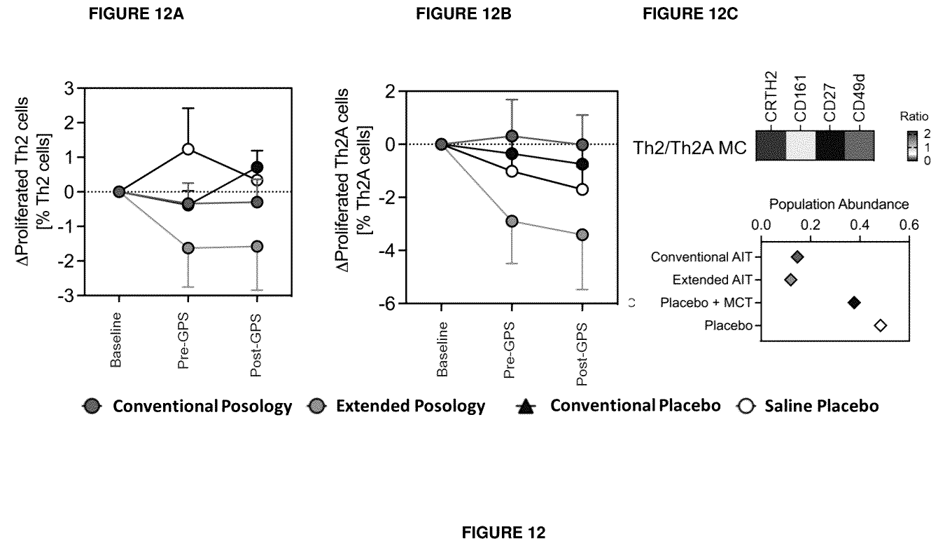

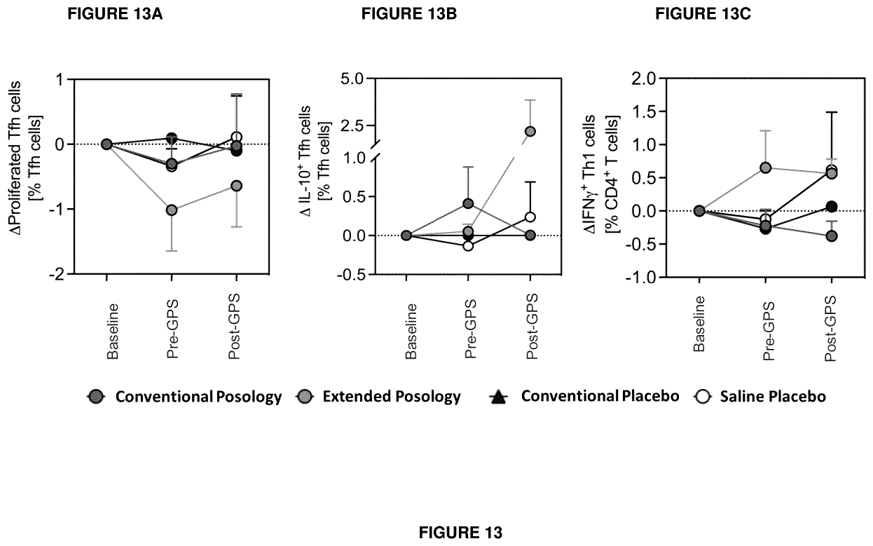

The greater treatment effect of the extended posology compared to the conventional posology was also consistent with the biomarker data:
FIGS. 11A-11C show that PQ Grass strongly induces nasal grass-specific IgA2, IgG and IgG4. Induction of grass-specific nasal IgA2, IgG and IgG4 after PQ Grass treatment persisting after grass season. The extended posology consistently showed most pronounced treatment effects on specific nasal lg.
FIGS. 12A-12C show that PQ Grass shows a reduction in allergen-driven Th2 and Th2A proliferation for extended posology.
FIGS. 13A-13C show that PQ Grass shows induction of immune deviation towards a type 1 response for the extended posology. There was a trend towards lower allergen-driven Tfh proliferation and a trend towards induction of protective IL-10⁺ Tfh cells and allergen-mediated IFN-g⁺ Th1 was observed for the extended posology.

Overall, the extended posology consistently demonstrated a more pronounced treatment effect on primary and secondary endpoints compared to the conventional posology in the G309 clinical trial, with no significant differences in the safety and tolerability profiles. A mild adverse event (AE) pattern was reported for both active treatment groups without any treatment related serious adverse event (SAE). The incidences of local and systemic AEs were similar between the two groups (extended and conventional posologies).

Conventional and extended regimens both showed a clinically relevant and statistically significant improvement in the primary outcome. For both regimens, predefined regulatory success criteria were reached due to the exceptionally large and clinically relevant treatment effect sizes observed in the primary and most secondary endpoints. The absolute effect size exceeded the minimal clinically relevant improvement of CSMS compared to placebo of 0.30 points as pre-agreed with European regulators by almost twofold (0.56 points) for the conventional regimen and even more substantially for the extended regimen (0.67 points). In addition, FDA success criteria (upper-limit of 95% CI below −10%) were also achieved for both regimens compared to placebo.

This trial was innovative in its evaluation of two different SCIT regimens using the same cumulative dose (27,600 SU) and established the extended regimen as the optimal choice to progress to the pivotal Phase III trial. An important contributing factor may be a better alignment of the extended regimen with a prophylactic vaccination concept, which generally considers longer periods between vaccinations. Using a 4-weekly maintenance dose instead of a once-weekly maintenance dose, the mechanical immunologic principle that germinal centers peak multiple weeks after antigen exposure (e.g. B-cell expansion, plasma cell generation, and T-follicular helper cell numbers) may have been better exploited.

Substantially lower primary endpoint effect sizes have been reported for pivotal Phase III studies of currently approved SLIT tablets. A single allergen SLIT tablet approved for the US market, reached a weighted average of 24.4% compared to placebo in TCS (Review, F.C. FDA clinical review—GRASTEK. BLA STN125473. Original BLA. 2013). The largest pivotal Phase III study (N=1301) reached a 20.5% difference to placebo (Maloney J, et al. Ann Allergy Asthma Immunol. 2014; 112: 146−153 e142). Additionally, for a 5-grass SLIT tablet, the weighted average of Phase III efficacy studies showed a 20.7% improvement compared to placebo (Review, F.C. ORALAIR, BLA STN125471. Original BLA. 2014).

Only a limited number of well-designed pivotal Phase III SCIT studies have been reported. A CSMS effect size of only 9.2% was reported for a recent pivotal Phase III house dust mite (HDM) SCIT study (EudraCT2016−000051−27. 2016). A Phase III peptide studies of HDM and cat, and a recent Depigoid Birch failed to reach their primary endpoint (NCT01620762, NCT02150343, and Novak N, et al. Clin Transl Allergy. 2022; 12:e12185). Moreover, a recent SCIT study applying peptide hydrolysates (gp-ASIT) did not meet clinically relevant effect sizes, with a mean reduction in CSMS of only 15.5% and 17.9% during the peak and entire GPS, respectively (Mosges R, et al. Allergy. 2018; 73: 1842−1850).

In conclusion, this trial demonstrated a large clinically relevant and statistically significant efficacy response of PQ Grass SCIT on the primary efficacy outcome. Unprecedented effect sizes for AIT of up to approximately 40% in the CSMS were achieved during the peak GPS compared to placebo using the extended regimen of only six pre-seasonal injections. This trial established the strong efficacy potential of PQ Grass using an optimized dose and an improved regimen.

### EMBODIMENTS

Various features and embodiments of the present invention will now be described with reference to the following numbered paragraphs (paras).

1. A method of treating allergic rhinitis and/or rhinoconjunctivitis in a subject, the method comprising administering to a subject in need thereof a therapeutically effective allergen-specific immunotherapy comprising or consisting of:
(a) a build-up phase consisting of three weekly subcutaneous injections of a composition comprising one or more antigens; and
(b) a maintenance phase consisting of three monthly subcutaneous injections of a composition comprising one or more antigens.

2. The method according to para 1, wherein the build-up phase consists of:
(i) a first weekly subcutaneous injection of a composition comprising one or more antigens in a dose of about 300 to about 1500 SU;
(ii) a second weekly subcutaneous injection of a composition comprising one or more antigens in a dose of about 2000 to about 4000 SU; and
(iii) a third weekly subcutaneous injection of a composition comprising one or more antigens in a dose of about 5000 to about 8000 SU.

3. The method according to para 1 or 2, wherein the second weekly subcutaneous injection is administered about 6 to about 9 days after the first subcutaneous injection and the third weekly subcutaneous injection is administered about 6 to about 9 days after the second weekly subcutaneous injection.

4. The method according to any preceding para, wherein the first weekly subcutaneous injection is a dose of about 900 SU, the second weekly subcutaneous injection is a dose of about 2700 SU, and the third weekly subcutaneous injection is a dose of about 6000 SU.

5. The method according to any preceding para, wherein the build-up phase and maintenance phase are separated by about 21 to about 35 days.

6. The method according to any preceding para, wherein the maintenance phase consists of:
(i) a first monthly subcutaneous injection of a composition comprising one or more antigens in a dose of about 5000 to about 8000 SU;
(ii) a second monthly subcutaneous injection of a composition comprising one or more antigens in a dose of about 5000 to about 8000 SU; and
(iii) a third monthly subcutaneous injection of a composition comprising one or more antigens in a dose of about 5000 to about 8000 SU.

7. The method according to any preceding para, wherein the second monthly subcutaneous injection is administered about 21 to about 35 days after the first subcutaneous injection and the third monthly subcutaneous injection is administered about 21 to about 35 days after the second monthly subcutaneous injection.

8. The method according to any preceding para, wherein the first monthly subcutaneous injection is a dose of about 6000 SU, the second monthly subcutaneous injection is a dose of about 6000 SU, and the third monthly subcutaneous injection is a dose of about 6000 SU.

9. A method of treating allergic rhinitis and/or rhinoconjunctivitis in a subject, the method comprising administering to a subject in need thereof a therapeutically effective allergen-specific immunotherapy comprising or consisting of:
(i) a first subcutaneous injection of a composition comprising one or more antigens in a dose of about 300 to about 1500 SU;
(ii) a second subcutaneous injection of a composition comprising one or more antigens in a dose of about 2000 to about 4000 SU, wherein the second subcutaneous injection is administered about 6 to about 9 days after the first subcutaneous injection;
(iii) a third subcutaneous injection of a composition comprising one or more antigens in a dose of about 5000 to about 8000 SU, wherein the third subcutaneous injection is administered about 6 to about 9 days after the second subcutaneous injection;
(iv) a fourth subcutaneous injection of a composition comprising one or more antigens in a dose of about 5000 to about 8000 SU, wherein the fourth subcutaneous injection is administered about 21 to about 35 days after the third subcutaneous injection;
(v) a fifth subcutaneous injection of a composition comprising one or more antigens in a dose of about 5000 to about 8000 SU, wherein the fifth subcutaneous injection is administered about 21 to about 35 days after the fourth subcutaneous injection; and
(vi) a sixth subcutaneous injection of a composition comprising one or more antigens in a dose of about 5000 to about 8000 SU, wherein the sixth subcutaneous injection is administered about 21 to about 35 days after the fifth subcutaneous injection.

10. The method according to para 9, wherein the first subcutaneous injection is a dose of about 900 SU, the second subcutaneous injection is a dose of about 2700 SU, the third subcutaneous injection is a dose of about 6000 SU, the fourth subcutaneous injection is a dose of about 6000 SU, the fifth subcutaneous injection is a dose of about 6000 SU, and the sixth subcutaneous injection is a dose of about 6000 SU.

11. The method according to any preceding para, wherein the final subcutaneous injection is administered within about 2 months prior to the predicted start of the allergy season, optionally wherein the final subcutaneous injection is administered within about 1 month to about 2 months prior to the predicted start of the allergy season or within about 1 week to about 7 weeks prior to the predicted start of the allergy season.

12. The method according to any preceding para, wherein each subcutaneous injection is about 0.5 mL to about 1.0 mL.

12. The method according to any preceding para, wherein each subcutaneous injection is to the lateral/posterior aspect of the middle third of the upper arm14. The method according to any preceding para, wherein the one or more antigens are derived from pollen.

15. The method according to any preceding para, wherein the one or more antigens are derived from Bent grass pollen, Foxtail pollen, Sweet vernal pollen, False oat pollen, Brome pollen, Dogstail pollen, Cocksfoot pollen, Fescue pollen, Yorkshire Fog pollen, Rye grass pollen, Timothy grass pollen, Meadow grass pollen and Cultivated Rye pollen.

16. The method according to any preceding para, wherein the one or more antigens are extracted from an antigen-containing material.

17. The method according to any preceding para, wherein the one or more antigens are diafiltered using a membrane with a 5 to 10 kDa molecular weight cut-off.

18. The method according to any preceding para, wherein the one or more antigens are modified with glutaraldehyde.

19. The method according to any preceding para, wherein the one or more antigens are adsorbed onto L-tyrosine.

20. The method according to any of paras 1 to 13, wherein each composition comprising one or more antigens comprises purified allergen extracts of pollen.

21. The method according to any of paras 1 to 13, wherein each composition comprising one or more antigens comprises equal amounts of purified allergen extracts of pollen from the following grasses: Bent grass, Foxtail, Sweet vernal, False oat, Brome, Dogstail, Cocksfoot, Fescue, Yorkshire Fog, Rye grass, Timothy grass, Meadow grass and Cultivated Rye.

22. The method according to para 20 or 21, wherein the allergens are modified into allergoids by treatment with glutaraldehyde.

23. The method according to any of paras 20 to 22, wherein the allergens are adsorbed onto L-tyrosine.

24. The method according to any preceding para, wherein each composition comprising one or more antigens comprises an amino acid.

25. The method according to para 24, wherein the amino acid is tyrosine, tryptophan or a derivative thereof.

26. The method according to para 24 or 25, wherein the amino acid is L-tyrosine.

27. The method according to para 26, wherein the L-tyrosine is in the form of microcrystalline tyrosine (MCT).

28. The method according to para 27, wherein the MCT has a median particle length of about 10 to about 100 microns, optionally about 10 to about 50 microns; and/or wherein the MCT has a median particle width of about 1 to about 10 microns, optionally about 2 about 6 microns.

29. The method according to any of paras 24 to 28, wherein each composition comprising one or more antigens comprises the amino acid in an amount of about 0.5% w/v to about 5% w/v, optionally about 2% w/v or about 3% w/v.

30. The method according to any preceding para, wherein each composition comprising one or more antigens comprises an adjuvant.

31. The method according to para 30, wherein the adjuvant is MPL, 3-DMPL or a derivative or salt thereof.

32. The method according to para 31, wherein the MPL, 3-DMPL or a derivative or salt thereof has a mean particle size of about 500 nm or less or about 200 nm or less.

33. The method according to any of paras 30 to 32, wherein each composition comprising one or more antigens comprises the adjuvant in an amount of about 25 μg/mL to about 100 μg/mL, optionally about 50 μg/mL.

34. The method according to any preceding para, wherein each composition comprising one or more antigens comprises L-tyrosine in an amount of about 2% w/v and MPL, 3-DMPL or a derivative or salt thereof in an amount of about 50 μg/mL.

35. The method according to any preceding para, wherein each composition comprising one or more antigens further comprises dipalmitoyl phosphatidylcholine (DPPC), phenol, sodium chloride, disodium phosphate dodecahydrate, sodium dihydrogen phosphate dihydrate and water.

36. The method according to any preceding para, wherein each composition comprising one or more antigens comprises DPPC in an amount of about 0.001 to about 0.01 mg/ml, optionally about 0.006 mg/ml.

37. The method according to any preceding para, wherein each composition comprising one or more antigens comprises phenol in an amount of about 1 to about 10 mg/ml, optionally about 5 mg/ml.

38. The method according to any preceding para, wherein each composition comprising one or more antigens comprises sodium chloride in an amount of from about 5 to about 20 mg/mL, optionally about 9.0 mg/mL.

39. The method according to any preceding para, wherein each composition comprising one or more antigens comprises disodium phosphate dodecahydrate and sodium dihydrogen phosphate dihydrate in an amount of about 1 to about 10 mg/mL, optionally about 4.0 mg/m L.

40. The method according to any preceding para, wherein each composition comprising one or more antigens is about pH 7.

41. The method according to any preceding para, wherein the rhinitis and/or rhinoconjunctivitis is caused by an IgE mediated allergy.

42. The method according to any preceding para, wherein the rhinitis and/or rhinoconjunctivitis is caused by an IgE mediated allergy against grass/rye, tree and/or weed pollen.

43. The method according to any preceding para, wherein the subject is a human.

44. The method according to any preceding para, wherein the subject is an adult, an adolescent or a child over the age of five.

45. The method according to any preceding para, wherein the subject has rhinitis and/or rhinoconjunctivitis caused by an IgE mediated allergy.

46. The method according to any preceding para, wherein the subject has rhinitis and/or rhinoconjunctivitis caused by an IgE mediated allergy against grass/rye, tree or weed pollen.

47. The method according to any preceding para, wherein the subject has been diagnosed with a positive skin prick test and/or specific IgE test.

48. A kit for treating allergic rhinitis and/or rhinoconjunctivitis in a subject, wherein the kit comprises:
(i) a first vial comprising about 0.5 to about 2.0 mL of a composition comprising one or more antigens in a dose of about 300 to about 1500 SU/mL;
(ii) a second vial comprising about 0.5 to about 2.0 mL of a composition comprising one or more antigens in a dose of about 2000 to about 4000 SU/mL;
(iii) a third vial comprising about 0.5 to about 2.0 mL of a composition comprising one or more antigens in a dose of about 5000 to about 8000 SU/mL;
(iv) a fourth vial comprising about 0.5 to about 2.0 mL of a composition comprising one or more antigens in a dose of about 5000 to about 8000 SU/mL;
(v) a fifth vial comprising about 0.5 to about 2.0 mL of a composition comprising one or more antigens in a dose of about 5000 to about 8000 SU/mL; and
(vi) a sixth vial comprising about 0.5 to about 2.0 mL of a composition comprising one or more antigens in a dose of about 5000 to about 8000 SU/m L.

49. The kit according to para 48, wherein the first vial comprises a dose of about 900 SU/mL, the second vial comprises a dose of about 2700 SU/mL, the third vial comprises a dose of about 6000 SU/mL, the fourth vial comprises a dose of about 6000 SU/mL, the fifth vial comprises a dose of about 6000 SU/mL, and the sixth vial comprises a dose of about 6000 SU/m L.

50. A kit for treating allergic rhinitis and/or rhinoconjunctivitis in a subject, wherein the kit comprises:
(i) a first syringe comprising about 0.5 mL to about 1.0 mL of a composition comprising one or more antigens in a dose of about 300 to about 1500 SU/mL;
(ii) a second syringe comprising about 0.5 mL to about 1.0 mL of a composition comprising one or more antigens in a dose of about 2000 to about 4000 SU/mL;
(iii) a third syringe comprising about 0.5 mL to about 1.0 mL of a composition comprising one or more antigens in a dose of about 5000 to about 8000 SU/mL;
(iv) a fourth syringe comprising about 0.5 mL to about 1.0 mL of a composition comprising one or more antigens in a dose of about 5000 to about 8000 SU/mL;
(v) a fifth syringe comprising about 0.5 mL to about 1.0 mL of a composition comprising one or more antigens in a dose of about 5000 to about 8000 SU/mL; and
(vi) a sixth syringe comprising about 0.5 mL to about 1.0 mL of a composition comprising one or more antigens in a dose of about 5000 to about 8000 SU/mL.

51. The kit according to para 50, wherein the first syringe comprises a dose of about 900 SU/mL, the second syringe comprises a dose of about 2700 SU/mL, the third syringe comprises a dose of about 6000 SU/mL, the fourth syringe comprises a dose of about 6000 SU/mL, the fifth syringe comprises a dose of about 6000 SU/mL, and the sixth syringe comprises a dose of about 6000 SU/mL.

52. The kit according to any of paras 48 to 51, wherein the one or more antigens are derived from pollen.

53. The kit according to any of paras 48 to 52, wherein the one or more antigens are derived from Bent grass pollen, Foxtail pollen, Sweet vernal pollen, False oat pollen, Brome pollen, Dogstail pollen, Cocksfoot pollen, Fescue pollen, Yorkshire Fog pollen, Rye grass pollen, Timothy grass pollen, Meadow grass pollen and Cultivated Rye pollen.

54. The kit according to any of paras 48 to 53, wherein the one or more antigens are extracted from an antigen-containing material.

55. The kit according to any of paras 48 to 54, wherein the one or more antigens are diafiltered using a membrane with a 5 to 10 kDa molecular weight cut-off.

56. The kit according to any of paras 48 to 55, wherein the one or more antigens are modified with glutaraldehyde.

57. The kit according to any of paras 48 to 56, wherein the one or more antigens are adsorbed onto L-tyrosine.

58. The kit according to any of paras 48 to 51, wherein each composition comprising one or more antigens comprises purified allergen extracts of pollen.

59. The kit according to any of paras 48 to 51, wherein each composition comprising one or more antigens comprises equal amounts of purified allergen extracts of pollen from the following grasses: Bent grass, Foxtail, Sweet vernal, False oat, Brome, Dogstail, Cocksfoot, Fescue, Yorkshire Fog, Rye grass, Timothy grass, Meadow grass and Cultivated Rye.

60. The kit according to para 58 or 59, wherein the allergens are modified into allergoids by treatment with glutaraldehyde.

61. The kit according to any of paras 58 to 60, wherein the allergens are adsorbed onto L-tyrosine.

62. The kit according to any of paras 48 to 61, wherein each composition comprising one or more antigens comprises an amino acid.

63. The kit according to para 62, wherein the amino acid is tyrosine, tryptophan or a derivative thereof.

64. The kit according to para 62 or 63, wherein the amino acid is L-tyrosine.

The kit according to para 64, wherein the L-tyrosine is in the form of microcrystalline tyrosine (MCT).

66. The kit according to para 65, wherein the MCT has a median particle length of about 10 to about 100 microns, optionally about 10 to about 50 microns; and/or wherein the MCT has a median particle width of about 1 to about 10 microns, optionally about 2 about 6 microns.

67. The kit according to any of paras 62 to 66, wherein each composition comprising one or more antigens comprises the amino acid in an amount of about 0.5% w/v to about 5% w/v, optionally about 2% w/v or about 3% w/v.

68. The kit according to any of paras 48 to 67, wherein each composition comprising one or more antigens comprises an adjuvant.

69. The kit according to para 68, wherein the adjuvant is MPL, 3-DMPL or a derivative or salt thereof.

70. The kit according to para 69, wherein the MPL, 3-DMPL or a derivative or salt thereof has a mean particle size of about 500 nm or less or about 200 nm or less.

71. The kit according to any of paras 68 to 70, wherein each composition comprising one or more antigens comprises the adjuvant in an amount of about 25 μg/mL to about 100 μg/mL, optionally about 50 μg/mL.

72. The kit according to any of paras 48 to 71, wherein each composition comprising one or more antigens comprises L-tyrosine in an amount of about 2% w/v and MPL, 3-DMPL or a derivative or salt thereof in an amount of about 50 μg/mL.

73. The kit according to any of paras 48 to 72, wherein each composition comprising one or more antigens further comprises dipalmitoyl phosphatidylcholine (DPPC), phenol, sodium chloride, disodium phosphate dodecahydrate, sodium dihydrogen phosphate dihydrate and water.

74. The kit according to any of paras 48 to 73, wherein each composition comprising one or more antigens comprises DPPC in an amount of about 0.001 to about 0.01 mg/ml, optionally about 0.006 mg/ml.

75. The kit according to any of paras 48 to 74, wherein each composition comprising one or more antigens comprises phenol in an amount of about 1 to about 10 mg/ml, optionally about 5 mg/ml.

76. The kit according to any of paras 48 to 75, wherein each composition comprising one or more antigens comprises sodium chloride in an amount of from about 5 to about 20 mg/mL, optionally about 9.0 mg/mL.

77. The kit according to any of paras 48 to 76, wherein each composition comprising one or more antigens comprises disodium phosphate dodecahydrate and sodium dihydrogen phosphate dihydrate in an amount of about 1 to about 10 mg/mL, optionally about 4.0 mg/m L.

78. The kit according to any of paras 48 to 77, wherein each composition comprising one or more antigens is about pH 7.

79. The kit according to any of paras 48 to 78, wherein the kit comprises instructions for a method of treating allergic rhinitis and/or rhinoconjunctivitis in a subject.

80. The kit according to para 79, wherein the method is defined according to any of paras 1 to 47.

1. A method of treating allergic rhinitis and/or rhinoconjunctivitis in a human subject, wherein the allergic rhinitis and/or rhinoconjunctivitis is caused by an IgE mediated allergy against pollen, the method comprising administering to a human subject in need thereof a therapeutically effective allergen-specific immunotherapy consisting of:
(i) a first subcutaneous injection of a composition comprising one or more pollen antigens in a dose of about 300 to about 1500 SU;
(ii) a second subcutaneous injection of a composition comprising one or more pollen antigens in a dose of about 2000 to about 4000 SU, wherein the second subcutaneous injection is administered about 6 to about 9 days after the first subcutaneous injection;
(iii) a third subcutaneous injection of a composition comprising one or more pollen antigens in a dose of about 5000 to about 8000 SU, wherein the third subcutaneous injection is administered about 6 to about 9 days after the second subcutaneous injection;
(iv) a fourth subcutaneous injection of a composition comprising one or more pollen antigens in a dose of about 5000 to about 8000 SU, wherein the fourth subcutaneous injection is administered about 21 to about 35 days after the third subcutaneous injection;
(v) a fifth subcutaneous injection of a composition comprising one or more pollen antigens in a dose of about 5000 to about 8000 SU, wherein the fifth subcutaneous injection is administered about 21 to about 35 days after the fourth subcutaneous injection; and
(vi) a sixth subcutaneous injection of a composition comprising one or more pollen antigens in a dose of about 5000 to about 8000 SU, wherein the sixth subcutaneous injection is administered about 21 to about 35 days after the fifth subcutaneous injection;
wherein each of the compositions comprising one or more pollen antigens comprises purified allergen extracts of pollen, wherein the allergens are modified by treatment with glutaraldehyde and adsorbed onto L-tyrosine, and wherein each of the compositions comprising one or more antigens comprises MPL, 3-DMPL or a derivative or salt thereof.

2. The method according to claim 1, wherein the final subcutaneous injection is administered within about 2 months prior to the predicted start of the pollen season.

3. The method according to claim 1, wherein each composition comprising one or more pollen antigens comprises equal amounts of purified allergen extracts of pollen from the following grasses: Bent grass, Foxtail, Sweet vernal, False oat, Brome, Dogstail, Cocksfoot, Fescue, Yorkshire Fog, Rye grass, Timothy grass, Meadow grass and Cultivated Rye, wherein the allergens are modified by treatment with glutaraldehyde and adsorbed onto L-tyrosine.

4. The method according to claim 1, wherein the L-tyrosine is in the form of microcrystalline tyrosine (MCT), wherein the MCT has a median particle length of about 10−100 microns and a median particle width of about 1−10 microns.

5. The method according to claim 1, wherein each composition comprising one or more pollen antigens comprises the L-tyrosine in an amount of about 0.5 to about 5% w/v.

6. The method according to claim 1, wherein each composition comprising one or more pollen antigens comprises the L-tyrosine in an amount of about 2% w/v.

7. The method according to claim 1, wherein the MPL, 3-DMPL or a derivative or salt thereof has a mean particle size of about 200 nm or less.

8. The method according to claim 1, wherein each composition comprising one or more pollen antigens comprises MPL, 3-DMPL or a derivative or salt thereof in an amount of about to about 100 μg/mL

9. The method according to claim 1, wherein each composition comprising one or more pollen antigens comprises MPL, 3-DMPL or a derivative or salt thereof in an amount of about 50 μg/mL

10. The method according to claim 1, wherein each composition further comprises dipalmitoyl phosphatidylcholine (DPPC), phenol, sodium chloride, disodium phosphate dodecahydrate, sodium dihydrogen phosphate dihydrate and water.

11. The method according to claim 1, wherein the rhinitis and/or rhinoconjunctivitis is caused by an IgE mediated allergy against grass/rye, tree and/or weed pollen.

12. The method according to claim 1, wherein the subject is an adult, an adolescent or a child over the age of five.

13. A kit for treating allergic rhinitis and/or rhinoconjunctivitis in a human subject, wherein the allergic rhinitis and/or rhinoconjunctivitis is caused by an IgE mediated allergy against pollen, wherein the kit comprises:
(i) a first vial comprising about 0.5 to about 2.0 mL of a composition comprising one or more pollen antigens in a dose of about 300 to about 1500 SU/mL;
(ii) a second vial comprising about 0.5 to about 2.0 mL of a composition comprising one or more pollen antigens in a dose of about 2000 to about 4000 SU/mL;
(iii) a third vial comprising about 0.5 to about 2.0 mL of a composition comprising one or more pollen antigens in a dose of about 5000 to about 8000 SU/mL;
(iv) a fourth vial comprising about 0.5 to about 2.0 mL of a composition comprising one or more pollen antigens in a dose of about 5000 to about 8000 SU/mL;
(v) a fifth vial comprising about 0.5 to about 2.0 mL of a composition comprising one or more pollen antigens in a dose of about 5000 to about 8000 SU/mL; and
(vi) a sixth vial comprising about 0.5 to about 2.0 mL of a composition comprising one or more pollen antigens in a dose of about 5000 to about 8000 SU/mL;
wherein each of the compositions comprising one or more pollen antigens comprises purified allergen extracts of pollen, wherein the allergens are modified by treatment with glutaraldehyde and adsorbed onto L-tyrosine, and wherein each of the compositions comprising one or more antigens comprises an adjuvant selected from MPL, 3-DMPL or a derivative or salt thereof.

14. The kit according to claim 13, wherein each composition comprising one or more pollen antigens comprises equal amounts of purified allergen extracts of pollen from the following grasses: Bent grass, Foxtail, Sweet vernal, False oat, Brome, Dogstail, Cocksfoot, Fescue, Yorkshire Fog, Rye grass, Timothy grass, Meadow grass and Cultivated Rye, wherein the allergens are modified by treatment with glutaraldehyde and adsorbed onto L-tyrosine.

15. The kit according to claim 13, wherein the L-tyrosine is in the form of microcrystalline tyrosine (MCT), wherein the MCT has a median particle length of about 10 to about 100 microns and a median particle width of about 1 to about 10 microns.

16. The kit according to claim 13, wherein each composition comprising one or more pollen antigens comprises the L-tyrosine in an amount of about 0.5 to about 5% w/v.

17. The kit according to claim 13, wherein the MPL, 3-DMPL or a derivative or salt thereof has a mean particle size of about 200 nm or less.

18. The kit according to claim 13, wherein each composition comprising one or more pollen antigens comprises MPL, 3-DMPL or a derivative or salt thereof in an amount of about 25 to about 100 μg/mL

19. The kit according to claim 13, wherein each composition comprising one or more pollen antigens comprises L-tyrosine in an amount of about 2% w/v and MPL, 3-DMPL or a derivative or salt thereof in an amount of about 50 μg/mL.

20. The kit according to claim 13, wherein each composition further comprises dipalmitoyl phosphatidylcholine (DPPC), phenol, sodium chloride, disodium phosphate dodecahydrate, sodium dihydrogen phosphate dihydrate and water.
